(19) United States
(12) Patent Application Publication
(10) Pub. No.: US 2002/0132813 A1
(43) Pub. Date: Sep. 19, 2002

(54) Heterocyclylalkylazole derivatives and their use as pesticides

(76) Inventors: Wolfgang Schaper, Diedorf (DE); Henricus Maria Martinus Bastiaans, Usingen (DE); Sven Harmsen, Liederbach (DE); Uwe Doller, Rodgau (DE); Daniela Jans, Bad Homburg (DE); Waltraud Hempel, Liederbach (DE); Ulrich Sanft, Eppstein (DE); Maria Theresia Thonessen, Heidesheim (DE)

Correspondence Address: FROMMER LAWRENCE & HAUG LLP, 745 Fifth Avenue, New York, NY, 10151, US

(21) Appl. No.: 09/923,197

(22) Filed: Aug. 6, 2001

(30) Foreign Application Priority Data
Aug. 8, 2000 (DE) 10039477.9

(51) Int. Cl. C07D 413/14; A61K 31/506; A61K 31/496; A61K 31/4545

(52) U.S. Cl. 514/252.19; 514/256; 544/295; 544/333; 546/193; 514/318

(57) ABSTRACT

Heterocyclylalkylazole derivatives and their use as pesticides Heterocyclylalkylazole derivatives of the formula (I)

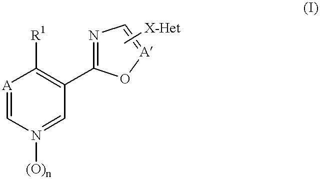

in which the symbols and indices are as defined in the description are highly suitable for controlling animal pests.

The invention relates to azolylalkylazole derivatives, to their preparation, and to their use for controlling animal pests, in particular arthropods such as insects and Acarina, and helminths.

It has already been disclosed that certain heterocyclylazoles exhibit an insecticidal action (WO-A 98/57969). However, since the ecological and economical demands made to modern insecticides are becoming more exacting all the time, for example with regard to toxicity, selectivity, application rates, formation of residues and advantageous ways of producing them, and since, moreover, problems may occur with, for example, resistances, there exists the constant task of developing novel insecticides which are advantageous over the known insecticides at least in some respects.

It has been found that compounds of the formula (I), if appropriate also as salts, have a good spectrum of action against animal pests while simultaneously being well tolerated by plants and having advantageous toxicological properties with regard to mammals and aquatic organisms.

Subject-matter of the invention are therefore heterocyclylalkylazole derivatives of the formula (I) and their salts

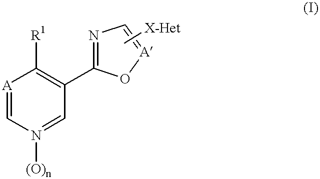

where the symbols and indices have the following meanings:

R₁ is (C₁-C₄)haloalkyl;

A, A′ are identical or different and are CH or N; where, in the event that A′=CH, the oxazole ring optionally has attached to it a further substituent;

n is 0 or 1;

X is a direct bond or an unbranched or branched (C₁-C₈)alkanediyl group in which a group

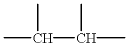

is optionally replaced by

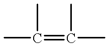

and/or a group —CH₂—CH₂— is optionally replaced by —C≡C— and in which a CH₂ group is optionally replaced by a carbonyl group or a heteroatom unit, preferably O, S(O)ₓ where x=0, 1 or 2, dimethylsilyl or —NR^{x}—;

R^{x} is H, (C₁-C₄)alkyl, (C₂-C₄)alkenyl, (C₂-C₄)alkynyl, (C₁-C₄)alkoxycarbonyl, (C₁-C₄)alkanoyl, (C₁-C₄)alkylsulfonyl, (C₁-C₄)alkoxy-(C₁-C₄)alkyl or (C₁-C₄)alkoxy;

Het is a group of the general formula (II)

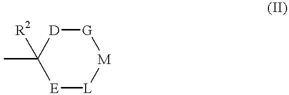

which is optionally mono- or polysubstituted and in which the symbols and indices have the following meanings:

D and E are identical or different and are in each case oxygen, S(O)ₚ where p=0, 1 or 2 or NR^{y},

R^{y} is R^{x}, aryl, aryl-(C₁-C₄)alkyl;

G and L are identical or different and are in each case CH₂ or a carbonyl group;

M is a direct bond, (C₁-C₃)alkanediyl, —CH═CH—,

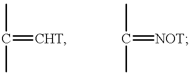

T is hydrogen, (C₁-C₈)alkyl, (C₁-C₈)haloalkyl, (C₂-C8)alkenyl, (C₂-C₈)alkynyl, (C₃-C₈)cycloalkyl, aryl, aryl-(C₁-C₄)alkyl;

it being possible for the aromatic ring systems mentioned for R^{y} and T to be unsubstituted or to be provided by up to three, in the case of fluorine also up to the maximum number of, identical or different substituents.

The symbols and indices in formulae (I) and (II) independently of one another have the following preferred meanings:

R¹ is preferably (C₁-C₄)fluoroalkyl, in particular CF₃.

A is preferably CH.

A′ is preferably N.

n is preferably 0.

X is preferably (C₁-C₄)alkanediyl, in particular —(CH₂)—, —(CH₂)₂— or —(CH₂)₃—.

D and E are preferably oxygen.

G and L are preferably CH₂.

M is preferably a direct bond, vinyl, vinylidene, —(CH₂)— or —(CH₂)₂—.

Substituents which are optionally present are preferably radicals R³, where

R³ is (C₁-C₈)alkyl, (C₂-C₈)alkenyl, (C₂-C₈)alkynyl, (C₃-C₈)cycloalkyl, (C₄-C₈)cycloalkenyl, aryl, heterocyclyl, halogen, hydroxyl, cyano, nitro, thiocyano, (C₁-C₈)alkoxy, (C₃-C₈)alkenyloxy, (C₃-C₈)alkynyloxy, (C₁-C₈)alkylthio, (C₁-C₈)alkylsulfinyl, (C₁-C₈)alkylsulfonyl, (C₃-C8)alkenylthio, (C₃-C₈)alkynylthio; a group COX where X is hydrogen, hydroxyl, (C₁-C₈)alkyl, (C₂-C₈)alkenyl, (C₂-C₈)alkynyl, (C₃-C₈)cycloalkyl, (C₁-C₈)alkoxy, amino, (C₁-C₈)alkylamino, (C₁-C₈)dialkylamino, heterocyclyl, aryl or aryl-(C₁-C₄)alkyl; a group NYZ where Y is hydrogen or (C₁-C₈)alkyl and Z is hydrogen, (C₁-C₈)alkyl, (C₃-C₈)cycloalkyl, (C₁-C₈)alkanoyl, (C₁-C₈)alkoxycarbonyl, (C₁-C₈)alkylsulfonyl, or hydrogen, where a saturated carbon unit in the R³ alkyl, cycloalkyl, cycloalkenyl, aliphatic heterocyclyl, alkenyl, alkynyl, alkoxy, alkenyloxy, alkynyloxy, alkylthio, alkylsulfinyl, aikylsulfonyl, alkenylthio, alkynylthio, alkylamino, dialkylamino, alkanoyl or alkoxycarbonyl group, can be replaced, so far as chemically meaningful, by a carbonyl group or a hetero atom unit such as oxygen, S(O)_{q} where q=0, 1 or 2, NR^{z}, where R^{z} has the meanings indicated above for R^{y}, or dimethylsilyi, and where additionally 3 to 8 atoms of these hydrocarbon radicals, which are optionally modified as above, may form a cycle and these hydrocarbon radicals, with or without the stated variations, may optionally be substituted by one or more, preferably up to three, in the case of fluorine up to the maximum number of, identical or different radicals selected from the series consisting of halogen, aryl, aryloxy, arylthio, cycloalkoxy, cycloalkylthio, heterocyclyl, heterocyclyloxy, heterocyclylthio, alkanoyl, cycloalkanoyl, haloalkanoyl, aroyl, arylalkanoyl, cycloalkylalkanoyl, heterocyclylalkanoyl, alkoxycarbonyl, haloalkoxycarbonyl, cycloalkoxycarbonyl, cycloalkylalkoxycarbonyl, arylalkoxycarbonyl, heterocyclyloxycarbonyl, heterocyclylalkoxycarbonyl, aryloxycarbonyl, heterocyclyloxycarbonyl, alkanoyloxy, haloalkanoyloxy, cycloalkanoyloxy, cycloalkylalkanoyloxy, aroyloxy, arylalkanoyloxy, heterocyclylalkanoyloxy, alkylsulfonyl, haloalkylsulfonyl, haloalkylsulfinyl, alkylsulfinyl, arylsulfinyl, arylsulfonyl, alkylsulfonyloxy, arylsulfonyloxy, amino, alkylamino, dialkylamino, alkanoylamino, alkanoylalkylamino, alkylsulfonylamino, alkylsulfonylalkylamino, hydroxyl, cyano, thiocyano or nitro,

where said cycloaliphatic, aromatic or heterocyclic ring systems may be unsubstituted or optionally provided with up to three, in the case of fluorine also up to the maximum number of, identical or different substituents; or two alkyl radicals on Het which are bound to different carbon atoms or to the same carbon atom together with these carbon atoms form a saturated or unsaturated 5- to 8-membered isocyclic ring which, instead of a saturated carbon unit, may also contain one or two oxygen or sulfur atoms or a group OCO and in which a single bond can be replaced by a double bond and which can optionally be substituted by up to four identical or different radicals, it being possible for these radicals to be (C₁-C₄)alkyl, halogen or (C₁-C₄)alkoxy.

If R³ denotes a substituent of the oxazole ring (A=CH), it preferably has the following meanings:

(C₁-C₄)alkyl, cyclopropyl, cyclobutyl, cyclobutenyl, vinyl, allyl, ethynyl, propargyl, methylcyclopropyl or cyclopropylmethyl, it being possible for the substituents mentioned to be optionally halogenated, preferably fluorinated;

in the event that A=CH, it is especially preferred that the oxazole ring is not further substituted.

If R³ denotes a substituent at the group Het, it preferably has the following meanings: (C₁-C₄)alkyl, cyclopropyl, cyclobutyl, cyclobutenyl, vinyl, allyl, ethynyl, propargyl, methylcyclopropyl or cyclopropylmethyl, it being possible for the substituents mentioned to be optionally halogenated, preferably fluorinated.

It is especially preferred that the group Het is unsubstituted or substituted by one or two (C₁-C₄)alkyl groups which can optionally have attached to them up to three fluorine atoms.

Unless defined in greater detail, aliphatic, aromatic or heterocyclic ring atoms are optionally provided with one or more, preferably up to three, in the case of fluorine also up to the maximum number of, substituents.

Preferred compounds of the formula (I) are those in which

R¹ is fluoroalkyl, in particular trifluoromethyl,

n is 0 and

A is CH.

More preferred compounds of the formula (I) are those in which

R¹ is trifluoromethyl,

n is 0 and

A is CH.

Even more preferred compounds of the formula (I) are those in which

R¹ is trifluoromethyl,

n is 0,

A is CH and

A′ is nitrogen.

Furthermore preferred are those compounds of the formula (I) in which

D and E are oxygen;

G and L are CH₂ and

M is a direct bond, vinyl, vinylidene, CH₂ or (CH₂)₂.

More preferred are those compounds of the formula (I) in which

D and E are oxygen;

G and L are CH₂;

M is a direct bond or CH₂ and, if appropriate,

R³ is (C₁-C₄)alkyl, cyclopropyl, cyclobutyl, cyclobutenyl, vinyl, allyl, ethynyl, propargyl, methylcyclopropyl or cyclopropylmethyl, it being possible for the substituents mentioned to be optionally halogenated, preferably fluorinated, and especially preferably

R³ is (C₁-C₄)alkyl, it being possible for the alkyl groups to have attached to them up to three fluorine atoms.

Furthermore preferred are the following groups of compounds of the formula (Ia) to (Ic) which can optionally be substituted by one or more radicals R³ and for which R⁴ is preferably (C₁-C₄)alkyl, (C₁-C₄)alkanoyl or (C₁-C₄)alkoxycarbonyl:

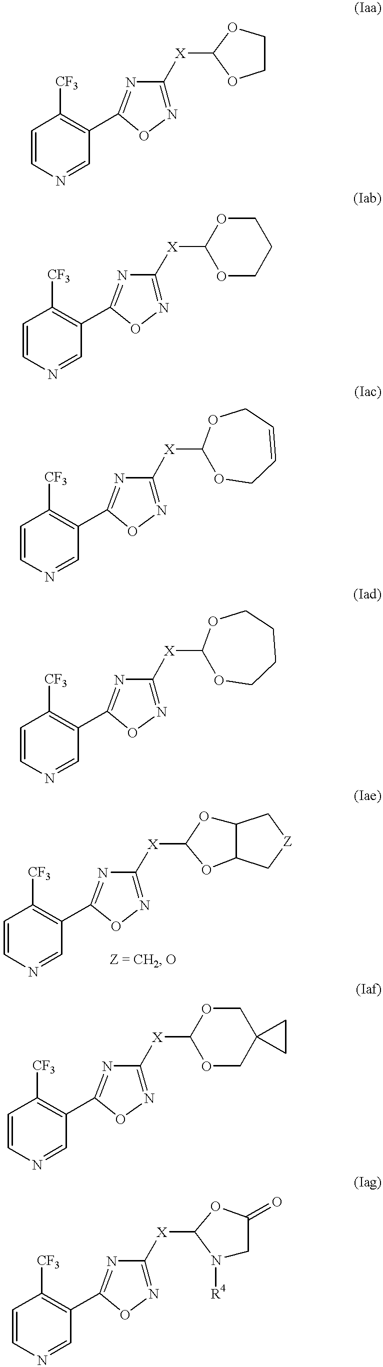

Especially preferred are compounds of the formulae (Iaa), (Iab), (Iac), (Iad), (Iae) and (Iaf).

In the above formula, “halogen” is to be understood as meaning a fluorine, chlorine, bromine or iodine atom;

the term “(C₁-C₄)alkyl” an unbranched or branched hydrocarbon radical having 1 to 4 carbon atoms, such as, for example, methyl, ethyl, propyl, isopropyl, 1-butyl, 2-butyl, 2-methylpropyl or tert-butyl radical;

the term “(C₁-C₈)alkyl” the abovementioned alkyl radicals and, for example, pentyl, 2-methylbutyl, tert-amyl, hexyl, heptyl, octyl or the 1,1,3,3-tetramethylbutyl radical;

the term “(C₁-C₄)haloalkyl” an alkyl group mentioned under the term “(C₁-C₄)alkyl” in which one or more hydrogen atoms are replaced by the abovementioned halogen atoms, preferably chlorine or fluorine, such as, for example, the trifluoromethyl group, 1-fluoroethyl group, the 2,2,2-trifluoroethyl group, the chloromethyl group, the fluoromethyl group, the difluoromethyl group, the 1,1,2,2-tetrafluoroethyl group or the difluorochloromethyl group;

the term “branched or unbranched (C₁-C₈)alkylene unit”, for example, the —(CH₂)—, —(CH₂)₂—, —CH(CH₃)—, —(CH₂)₃—, —CH₂CH(CH₃)—, CH(CH₃)—CH₂, —(CH₂)₄, —H₂—CH(CH₃)CH₂—, —(CH₂)₂—CH(CH₃)—, —(CH₂)₅—, —(CH₂)₆—, —(CH₂)₇— or —(CH₂)₈ unit;

the term “aryl-(C₁-C₄)alkyl”, for example, the benzyl, 2-phenylethyl, α-methylbenzyl, 3-phenylpropyl, 2-phenylpropyl, α-ethylbenzyl, 4-phenylbutyl or 1-phenylbutyl group.

The terms “alkenyl” and “alkynyl” with a prefix for the range of carbon atoms denote a straight-chain or branched hydrocarbon radical with a number of carbon atoms corresponding to this prefix which contains at least one multiple bond, it being possible for the latter to be located in any position of the unsaturated radical in question.

The term “(C₃-C₈)cycloalkyl” is to be understood as meaning the cyclopropyl, cyclobutyl, cyclopentyl, cyclohexyl, cycloheptyl or cyclooctyl group; the term “aryl” an isocyclic aromatic radical having preferably 6 to 14, in particular 6 to 12, carbon atoms, for example phenyl, naphthyl or biphenylyl, preferably phenyl; the term “heterocyclyl” a heteroaromatic or heteroaliphatic ring system, “heteroaromatic ring system” being understood as meaning an aryl radical in which at least one CH group is replaced by N and/or at least two adjacent CH groups are replaced by S, NH or O, for example a radical of thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]-thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine or 4H-quinolizine;

the term “heteroaliphatic ring system” a (C₃-C₈)cycloalkyl radical in which at least one carbon unit is replaced by O, S, SO, SO₂ or a group NR^{v} and R^{v} has the meanings indicated above for R^{y};

the term “(C₄-C₈)cycloalkenyl” the cyclobutenyl, cyclopentenyl, cyclohexenyl, cycloheptenyl or cyclooctenyl group;

the term “(C₁-C₄)alkoxy” an alkoxy group whose hydrocarbon radical is as defined for the term “(C₁-C₄)alkyl”;

the term “(C₁-C₈)alkoxy” an alkoxy group whose hydrocarbon radical is as defined for the term “(C₁-C₈)alkyl”;

the term “(C₃-C₈)alkenyloxy”, for example, the allyloxy, crotyloxy, 3-buten-1 -yloxy, 1 -penten-3-yloxy, 1 -penten-4-yloxy or the 3-penten-2-yloxy group;

the term “(C₃-C₈)alkynyloxy”, for example, the propargyloxy, 1-butyn-3-yloxy, 2-butyn-1 -yloxy or the 3-butyn-1 -yloxy group;

the term “(C₁-C₄)alkylthio” the alkylthio group whose alkyl radical is as defined for the term “(C₁-C₄)alkyl”;

the term “(C₁-C₈)alkylthio” an alkylthio group whose alkyl radicals have the meanings given under the term “(C₁-C₈)alkyl”;

the term “(C₁-C₄)alkylsulfinyl” for example the methyl-, ethyl-, propyl-, isopropyl-, butyl-, isobutyl-, sec-butyl- or the tert-butylsulfinyl group;

the term “(C₁-C₈)alkylsulfinyl” for example the abovementioned groups and, for example, the pentylsulfinyl or the octylsulfinyl group;

the term “(C₁-C₄)alkylsulfonyl” for example the methyl-, ethyl-, propyl-, isopropyl-, butyl-, sec-butyl-, isobutyl-or tert-butylsulfonyl group;

the term “(C₁-C₈)alkylsulfonyl” the abovementioned radicals and, for example, the pentyl-, hexyl-, heptyl- or octylsulfonyl group;

the term “(C₃-C₈)alkenylthio” for example the allylthio-, crotylthio-, 3-buten-1-ylthio- or the 3-penten-2-ylthio group;

the term “(C₃-C₈)alkynylthio” for example the propargylthio-, 1-butyn-3-ylthio- or the 3-butyn-1-ylthio group;

the term “(C₁-C₄)alkylamino” for example the methylamino-, ethylamino-, propylamino-, isopropylamino-, butylamino-, isobutylamino-, sec-butylamino- or the tert-butylamino group;

the term “(C₁-C₈)alkylamino” for example the abovementioned groups and, for example, the pentylamino-, n-octylamino or the tert-octylamino group;

the term “(C₁-C₄)dialkylamino” for example the dimethylamino-, N-methyl-N-ethylamino-, diethylamino-, dipropylamino or the dibutylamino group, but also cyclic systems such as, for example, the pyrrolidino or piperidino group, and also those cyclic systems which contain a heteroatom such as, for example, the morpholino, thiomorpholino or piperazino group;

the term “(C₁-C₈)dialkylamino”, for example the abovementioned groups and, for example, the N-methyl-N-pentyl or the N-methyl-N-octyl group;

the term “(C₁-C₄)alkanoyl” for example the formyl, acetyl, propionyl, 2-methylpropionyl or the butyryl group;

the term “(C₁-C₈)alkanoyl” for example the abovementioned groups and, for example, the valeroyl, pivaloyl, hexanoyl, heptanoyl or octanoyl group;

the term “(C₁-C₄)alkoxycarbonyl” for example the methoxycarbonyl, ethoxycarbonyl, propoxycarbonyl, butoxycarbonyl, isopbutoxy or the tert-butoxycarbonyl group;

the term “(C₁-C₈)alkoxycarbonyl” for example the abovementioned groups and, for example, the pentyloxycarbonyl, hexyloxycarbonyl or octyloxycarbonyl group;

the term “(C₁-C₄)alkoxy(C₁-C₄)alkyl” for example the methoxymethyl, methoxyethyl, ethoxyethyl, propoxymethyl or the butoxymethyl group;

the term “(C₁-C₄)trialkylsilyl” for example the dimethylethylsilyl, dimethyltert-butylsilyl or preferably the trimethylsilyl group.

The substituents with which the various aliphatic, aromatic and heterocyclic ring systems can be provided, for example, halogen, nitro, cyano, amino, (C₁-C₄)alkylamino, di-(C₁-C₄)alkylamino, (C₁-C₄)alkyl, (C₂-C₄)alkenyl, (C₂-C₄)alkynyl, (C₃-C₈)cycloalkyl, (C₁-C₄)trialkylsilyl, (C₁-C₄)alkoxy, (C₁-C₄)alkoxy-(C₁-C₄)alkyl, (C₁-C₂)alkoxy-[CH₂CH₂O]_{0,1,2}-ethoxy, (C₁-C₄)alkylthio, (C₁-C₄)alkylsulfinyl, (C₁-C₄)alkylsulfonyl, phenyl, benzyl, phenoxy, halophenoxy, (C₁-C₄)alkylphenoxy, (C₁-C₄)alkoxyphenoxy, phenylthio, heterocyclyl, heterocyclylthio or heterocyclyloxy, it being possible for one or more, in the case of fluorine also up to the maximum number of, hydrogen atoms in the alkyl, alkenyl and alkynyl radicals and the radicals derived therefrom to be replaced by halogen, preferably chlorine or fluorine; where, in the event that these substituents are (C₁-C₄)alkyl, they may also be linked cyclically and where one ortwo aliphatic carbon units in these fused ring systems, such as, for example, the indane, di-, tetra- or decahydronaphthyl or benzocycloheptane system, may be replaced by heteroatom units such as oxygen or sulfur and where one or more, in the case of fluorine also up to the maximum number of, hydrogen atoms on the aliphatic carbon atom units can be replaced by halogen or (C₁-C₄)alkyl.

Preference is given to halogen, (C₁-C₄)alkyl, (C₁-C₄)trialkylsilyl, (C₁-C₄)alkoxy, (C₁-C₄)alkoxy-(C₁-C₄)alkyl, (C₁-C₄)alkylthio, (C₁-C₄)alkylsulfinyl, (C₁-C₄)alkylsulfonyl, the alkyl groups in said radicals possibly being optionally substituted by one or more halogen atoms, preferably fluorine.

The definition that where a saturated carbon unit in the R³ alkyl, cycloalkyl, cycloalkenyl, aliphatic heterocyclyl, alkenyl, alkynyl, alkoxy, alkenyloxy, alkynyloxy, alkylthio, alkylsulfinyl, alkylsulfonyl, alkenylthio, alkynylthio, alkylamino, dialkylamino, alkanoyl or alkoxycarbonyl group, can be replaced, so far as chemically meaningful, by a carbonyl group or a heteroatom unit such as oxygen, S(O)_{q} where q=0, 1 or 2, NR^{z}, where R^{z} has the meanings indicated above for R^{y}, or dimethylsilyl, and where additionally 3 to 8 atoms of these hydrocarbon radicals, which are optionally modified as above, may form a cycle and these hydrocarbon radicals, with or without the stated variations, may optionally be substituted by one or more, preferably up to three, in the case of fluorine up to the maximum number of, identical or different radicals selected from the series consisting of halogen, aryl, aryloxy, arylthio, cycloalkoxy, cycloalkylthio, heterocyclyl, heterocyclyloxy, heterocyclylthio, alkanoyl, cycloalkanoyl, haloalkanoyl, aroyl, arylalkanoyl, cycloalkylalkanoyl, heterocyclylalkanoyl, alkoxycarbonyl, haloalkoxycarbonyl, cycloalkoxycarbonyl, cycloalkylalkoxycarbonyl, arylalkoxycarbonyl, heterocyclyloxycarbonyl, heterocyclylalkoxycarbonyl, aryloxycarbonyl, heterocyclyloxycarbonyl, alkanoyloxy, haloalkanoyloxy, cycloalkanoyloxy, cycloalkylalkanoyloxy, aroyloxy, arylalkanoyloxy, heterocyclylalkanoyloxy, alkylsulfonyl, haloalkylsulfonyl, haloalkylsulfinyl, alkylsulfinyl, arylsulfinyl, arylsulfonyl, alkylsulfonyloxy, arylsulfonyloxy, amino, alkylamino, dialkylamino, alkanoylamino, alkanoylalkylamino, alkylsulfonylamino, alkylsulfonylalkylamino, hydroxyl, cyano, thiocyano or nitro, is furthermore to be understood as meaning, for example,

alkoxyalkyl radicals such as, for example, the methoxymethyl, methoxyethyl or ethoxyethyl group; or

alkoxyalkoxyalkyl radicals such as, for example, the methoxy- or the ethoxyethoxyethyl group; or

alkylthioalkyl radicals such as, for example, the methyl- or the ethylthioethyl group; or

alkylsulfonylalkyl radicals such as, for example, the methylsulfonylmethyl, methylsulfonylethyl or the ethylsulfonylmethyl group; or

alkylsulfinylalkyl radicals such as, for example, the methyl or ethylsulfinylethyl group; or

alkyldimethylsilylalkyl radicals such as, for example, the trimethylsilylmethyl or the trimethylsilylethyl group; or

alkyldimethylsilyl radicals such as, for example, the trimethylsilyl, ethyldimethylsilyl, tert-butyldimethylsilyl or the octyldimethylsilyl group; or

cycloalkyldimethylsilyl radicals such as, for example, the cyclohexyldimethylsilyl group; or

aryldimethylsilyl radicals such as, for example, the phenyldimethylsilyl group; or

arylalkyldimethylsilyl radicals such as, for example, the benzyldimethylsilyl or the phenylethyldimethylsilyl group; or

alkanoylalkyl radicals such as, for example, the acetylmethyl or the pivaloylmethyl group; or

alkanoylaminoalkyl radicals such as, for example, the acetylaminomethyl group or alkylsulfonylaminoalkyl radicals such as, for example, the methylsulfonylaminomethyl group; or

cycloalkanoylalkyl radicals such as, for example, the cyclopropylcarbonylmethyl or the cyclohexylcarbonylmethyl group; or

haloalkanoylalkyl radicals such as, for example, the trifluoro- or trichloroacetylmethyl group; or aroylalkyl radicals such as, for example, the benzoyl-, naphthoyl or phenylacetylmethyl group; or

heterocyclylcarbonylalkyl radicals such as, for example, the thienyl- or pyridylacetylmethyl group; or

arylalkyl radicals such as, for example, the benzyl, the 2-phenylethyl, the 1-phenylethyl, the 1-methyl-1-phenylethyl group, the 3-phenylpropyl, the 4-phenylbutyl group, the 2-methyl-2-phenylethyl group or the 1-methyl- or 2-methyinaphthyl group; or

heterocyclylalkyl radicals such as, for example, the thienylmethyl, pyridylmethyl, furfuryl, tetrahydrofurfuryl, tetrahydropyranylmethyl or the 1,3-dioxolan-2-ylmethyl group; or aryloxyalkyl radicals such as, for example, the phenoxymethyl or naphthoxymethyl group; or

cycloalkyl radicals, monocyclic such as, for example, the cyclopropyl, cyclobutyl, cyclopentyl, cyclohexyl, cycloheptyl or cyclooctyl radical, bicyclic such as, for example, the norbornyl radical or the bicyclo[2,2,2]octyl radical, or fused, such as the decahydronaphthyl radical;

alkylcycloalkyl radicals such as, for example, the 4-methyl- or the 4-tert-butylcyclohexyl group or the 1 -methylcyclopropyl, -cyclobutyl, -cyclopentyl or -cyclohexyl group;

cycloalkylalkyl radicals such as, for example, the cyclohexylmethyl or -ethyl group;

cycloalkylene radicals, monocyclic such as, for example, the cyclopentenyl, cyclohexenyl, cycloheptenyl or cyclooctenyl radical, bicyclic such as, for example, the norbornenyl or the bicyclo[2,2,2]octenyl radical, or fused, such as the various dihydro- or tetrahydronaphthyl radicals;

cycloalkylenealkyl radicals such as, for example, the 1-cyclohexenylmethyl or -ethyl radical;

or else haloalkyl derivatives of the corresponding group, such as, for example, haloalkyl, haloalkoxyalkyl, alkoxyhaloalkoxy, haloalkylcycloalkyl or halocycloalkyl radicals.

Moreover, the term “that two alkyl radicals at Het which are bonded to different carbon atoms or to the same carbon atom together with these carbon atoms form a saturated or unsaturated 5- to 8-membered isocyclic ring which, instead of a saturated carbon unit, can also contain one or two oxygen or sulfur atoms or a group OCO and in which a single bond can be replaced by a double bond and which can optionally be substituted by up to four identical or different radicals and these radicals can be (C₁-C₄)alkyl, halogen or (C₁-C₄)alkoxy” is to be understood as meaning that these radicals R³, when they are attached to adjacent carbon atoms, are linked to give a fused cyclopentano, cyclohexano, tetrahydrofurano or butyrolactono ring or, when they are attached to the same carbon atom, together with the aliphatic heterocycle form a spiro system, or, when they are attached to the next but one carbon atom, they are linked to form a bicyclic system.

What has been said above applies analogously to homologs or their derived radicals.

The present invention relates to the compounds of the formula (I) in the form of the free base or of an acid addition salt. Acids which can be used for salt formation are inorganic acids such as hydrochloric acid, hydrobromic acid, nitric acid, sulfuric acid, phosphoric acid, or organic acids such as formic acid, acetic acid, propionic acid, malonic acid, oxalic acid, fumaric acid, adipic acid, stearic acid, oleic acid, methanesulfonic acid, benzenesulfonic acid or toluenesulfonic acid.

Preference is given to the salts which are accepted and customary in the field of pest control.

Some of the compounds of the formula (I) have one or more asymmetric carbon atoms or stereoisomers on double bonds. Enantiomers or diastereomers are therefore possible. The invention encompasses not only the pure isomers, but also their mixtures. The diastereomer mixtures can be resolved into the components by customary methods, for example by selective crystallization from suitable solvents or by chromatography. Racemates can be resolved by customary methods to give the enantiomers, for example by salt formation with a chiral enantiomerically pure acid, separation of the diastereomeric salts and setting free the pure enantiomers by means of a base.

The present invention also relates to processes for the preparation of compounds of the formula (I).

To prepare compounds of the formula (I) in which A, R¹, X, n and Het have the meanings indicated for formula (I) and A′ is nitrogen, a procedure is followed for example in which a carboxylic acid of the formula (ill)

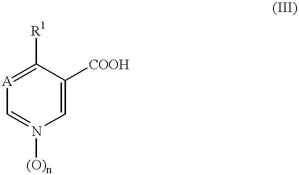

in which

A, R¹ and n have the meanings indicated above for formula (I) are reacted, in the form of an activated derivative of this acid, with a compound of the formula (IV) in which Het and X have the meanings indicated above for formula (I)

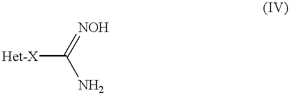

in the presence of a base.

An example of an activated derivative which can be employed is an acyl halide, an ester or an anhydride. Bases which are suitable are, for example, amines such as triethylamine, diisopropylethylamine, pyridine or lutidine, alkali metal hydroxides, alkali metal alkoxides such as sodium methoxide or potassium tert-butoxide, or alkyl metal compounds such as butyllithium.

The reaction described can be carried out as a one-step process or as a two-step process, depending on the choice of the conditions, given, as intermediates, compounds of the formula (V)

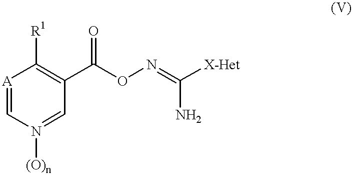

in which A, R¹, n, X and Het are as defined above for the formula (I).

Compounds of the formula (V) can be cyclized to give the 1,2,4-oxadiazoles by heating in an inert solvent at temperatures of up to 180° C. Compounds of the formula (V) can also be obtained directly from the acid of the formula (III) and amidoximes of the formula (IV) by using a dehydrating reagent such as dicyclohexylcarbodiimide, 1 -ethyl-3-(3-dimethylaminopropyl)carbodiimide or N,N′-carbonyidiimidazole.

The invention likewise relates to intermediates of the formula (V).

Both acids of the formula (III) and amidoximes of the formula (IV) are known or can be prepared by processes known from the literature (see, for example, Houben-Weyl, Methoden der Organischen Chemie [Methods in Organic Chemistry], Volume X/4, pages 209-212; EP-A 0 580 374; G. F. Holland, J. N. Pereira, J. Med. Chem. 1967,10,149).

The most preferred compounds of the formula (I) where D and E are oxygen and G and L are CH₂ can furthermore be prepared by a process in which a compound of the formula (VI)

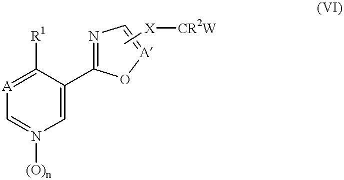

in which

A, A′, R¹, n and X have the meanings indicated above for formula (I) and (II), R² is H or R³, and W is (═O) or (OR⁶)₂, where R⁶ is (C₁-C₄)alkyl, are reacted with a compound of the formula (VII)

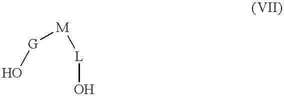

in which

G and L are CH₂, M and m have the meanings indicated for formula (II), in the presence of an acid to give the end products of the formula I.

The above-described acetalization of the reaction is known in principle. It is generally carried out in a temperature range of 20-200°, preferably between 60° and 150°, in the presence of an acidic dehydration catalyst, solid or in an inert solvent. Suitable catalysts are, for example, hydrochloric acid, sulfuric acid, phosphoric acid, sodium hydrogen sulfate, sulfonic acids such as methane- or toluenesulfonic acid, phosphorus(V) oxide, iron(III) chloride, zinc chloride, anhydrous copper sulfate, iodine or else acidic ion exchangers such as, for example, ®Amberlite IR-120. The water formed during the reaction when W is (═O) is expediently removed from the reaction mixture by distillation, if appropriate under reduced pressure, or by azeotropic distillation using an entrainer. Suitable entrainers are, for example, benzene, toluene, xylene or petroleum ether.

The starting materials of the formula (VI) are known (WO-A-98/57969) or can be prepared analogously to known processes. This also applies analogously to the diols of the formula (VII).

Collections of compounds of the formula (I) which can be synthesized by the abovementioned scheme may also be prepared in parallel, and this may be effected manually or in a semiautomated or fully automated fashion. For example, it is possible to automate the procedure of the reaction, work-up or purification of the products or intermediates. In total, this is to be understood as a procedure as is described, for example, by S. H. DeWitt in “Annual Reports in Combinatorial Chemistry and Molecular Diversity: Automated synthesis”, Volume 1, Verlag Escom 1997, pages 69 to 77.

A series of commercially available apparatuses may be used for the parallel procedure of the reaction and work-up as are offered, for example, by Stem Corporation, Woodrolfe Road, Tollesbury, Essex, CM98SE, England, or H+P Labortechnik GmbH, Bruckmannring 28, 85764 Oberschleiβheim, Germany, or Radleys, Shirehill, Saffron Walden, Essex, England. Equipment which is available for the parallel purification of compounds of the formula (I) or of intermediates obtained during the preparation are, inter alia, chromatography apparatuses, for example those of ISCO, Inc., 4700 Superior Street, Lincoln, Nebr. 68504, USA.

The apparatuses mentioned result in a modular procedure, in which the individual passes are automated, but manual operations must be carried out between the passes. This can be circumvented by employing partially or fully integrated automation systems in which the automation modules in question are operated by, for example, robots. Such automation systems can be obtained, for example, from Zymark Corporation, Zymark Center, Hopkinton, Mass. 01748, USA.

In addition to the methods described here, compounds of the formula (I) can be prepared completely or partially by solid-phase-aided methods. To this end, individual intermediates or all intermediates of the synthesis or of a synthesis adapted to suit the procedure in question are bound to a synthetic resin. Solid-phase-aided synthetic methods are described extensively in the specialist literature, for example Barry A. Bunin in “The Combinatorial Index”, Verlag Academic Press, 1998.

The use of solid-phase-aided synthetic methods allows a series of protocols known from the literature, which, in turn, can be carried out manually or in an automated fashion. For example, the tea-bag method (Houghten, U.S. Pat. No. 4,631,211; Houghten et al., Proc. Natl. Acad. Sci, 1985, 82, 5131-5135) can be partially automated using products by IRORI, 11149 North Torrey Pines Road, La Jolla, Calif. 92037, USA. Solid-phase-aided parallel syntheses are successfully automated by, for example, apparatuses by Argonaut Technologies, Inc., 887 Industrial Road, San Carlos, Calif. 94070, USA or MultiSynTech GmbH, Wullener Feld 4, 58454 Wiften, Germany.

The preparation in accordance with the processes described herein yields compounds of the formula (I) in the form of substance collections, which are termed libraries. The present invention also relates to libraries comprising at least two compounds of the formula (I).

The compounds of the formula (I) are suitable for controlling animal pests, in particular insects, arachnids, helminths and molluscs, very particularly preferably for controlling insects and arachnids found in agriculture, in livestock production, in forests, in the protection of stored products and materials, and in the hygiene sector, while being well tolerated by plants and having a favorable toxicity to warm-blooded species. They are active against normally-sensitive and resistant species and against all or individual developmental stages. The abovementioned pests include:

From the order of the Acarina, for example, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Eotetranychus spp., Oligonychus spp., Eutetranychus spp.

From the order of the Isopoda, for example, Oniscus aselus, Armadium vulgare, Porcellio scaber.

From the order of the Diplopoda, for example, Blaniulus guftulatus.

From the order of the Chilopoda, for example, Geophilus carpophagus, Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Blatta orientalis, Periplaneta americana, Leucophaea maderae, Blattella germanica, Acheta domesticus, Gryllotalpa spp., Locusta migratoria migratorioides, Melanoplus differentialis, Schistocerca gregaria.

From the order of the Isoptera, for example, Reticulitermes spp.

From the order of the Anoplura, for example, Phylloxera vastatrix, Pemphigus spp., Pediculus humanus corporis, Haematopinus spp., Linognathus spp.

From the order of the Mallophaga, for example, Trichodectes pp., Damalinea spp.

From the order of the Thysanoptera, for example, Hercinothrips femoralis, Thrips tabaci.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Doralis fabae, Doralis pomi, Eriosoma lanigerum, Hyalopterus arundinis, Macrosiphum avenae, Myzus spp., Phorodon humuli, Rhopalosiphum padi, Empoasca spp., Euscelus bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Cheimatobia brumata, Lithocolletis blancardella, Hyponomeuta padella, Plutella maculipennis, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Laphygma exigua, Mamestra brassicae, Panolis flammea, Prodenia litura, Spodoptera spp., Trichoplusia ni, Carpocapsa pomonella, Pieris spp., Chilo spp., Pyrausta nubilalis, Ephestia kuehniella, Galleria mellonella, Cacoecia podana, Capua reticulana, Choristoneura fumiferana, Clysia ambiguella, Homona magnanima, Tortrix viridana.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylloides chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrynchus assimilis, Hypera postica, Dermestes spp., Trogoderma, Anthrenus spp., Attagenus spp., Lyctus spp., Meligethes aeneus, Ptinus spp., Niptus hololeucus, Gibbium psylloides, tribolium spp., Tenebrio molitor, Agriotes spp., Conoderus spp., Melolontha melolontha, Amphimallon soistitialis, Costelytra zealandica.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hypobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa.

From the order of the Siphonaptera, for example, Xenopsylla cheopsis, Ceratophyllus spp.

From the order of the Arachnida, for example, Scorpio maurus, Latrodectus mactans.

From the class of the helminths, for example, Haemonchus, Trichostrongulus, Ostertagia, Cooperia, Chabertia, Strongyloides, Oesophagostomum, Hyostrongulus, Ancylostoma, Ascaris, Heterakis and Fasciola.

From the class of the Gastropoda, for example, Deroceras spp., Arion spp., Lymnaea spp., Galba spp., Succinea spp., Biomphalaria spp., Bulinus spp., Oncomelania spp.

From the class of the Bivalva, for example, Dreissena spp.

The plant-parasitic nematodes which can be controlled in accordance with the invention include, for example, the root-parasitic soil-dwelling nematodes such as, for example, those of the genera Meloidogyne (root-knot nematodes such as Meloidogyne incognita, Meloidogyne hapla and Meloidogyne javanica), Heterodera and Globodera (cyst nematodes such as Globodera rostochiensis, Globodera pallida, Heterodera trifolii) and of the genera Radopholus such as Radopholus similis, Pratylenchus such as Pratylenchus neglectus, Pratylenchus penetrans and Pratylenchus curvitatus;

Tylenchulus such as Tylenchulus semipenetrans, Tylenchorhynchus, such as Tylenchorhynchus dubius and Tylenchorhynchus claytoni, Rotylenchus such as Rotylenchus robustus, Heliocotylenchus such as Haliocotylenchus multicinctus, Belonoaimus such as Belonoaimus longicaudatus, Longidorus such as Longidorus elongatus, Trichodorus such as Trichodorus primitivus and Xiphinema such as Xiphinema index.

The nematode genera Ditylenchus (stem parasites such as Ditylenchus dipsaci and Ditylenchus destructor), Aphelenchoides (foliar nematodes such as Aphelenchoides ritzemabosi) and Anguina (seed nematodes such as Anguina tritici) can also be controlled with the compounds according to the invention.

The invention also relates to compositions, for example crop protection compositions, preferably to insecticidal, acaricidal, ixodicidal, nematicidal, molluscicidal or fungicidal compositions, especially preferably insecticidal and acaricidal compositions, which comprise one or more compounds of the formula (I) in addition to suitable formulation auxiliaries.

The compositions according to the invention generally contain 1 to 95% by weight of the active substances of the formula (I).

To prepare the compositions according to the invention, the active substance and the further additives are combined and formulated to give a suitable use form.

The invention also relates to compositions, in particular to insecticidal and acaricidal compositions, which comprise the compounds of the formula (I) in addition to is suitable formulation auxiliaries.

In general, the active substances of the formula (I) amount to 1 to 95% by weight of the compositions according to the invention. The latter can be formulated in various ways, depending on the biological and/or chemico-physical parameters which prevail. The following are therefore examples of possibilities of formulation: Wettable powders (WP), emulsifiable concentrates (EC), aqueous solutions (SL), emulsions, sprayable solutions, oil- or water-based dispersions (SC), suspoemulsions (SE), dusts (DP), seed-dressing materials, granules in the form of microgranules, spray granules, coated granules and adsorption granules, water-dispersible granules (WG), ULV formulations, microcapsules, waxes or baits.

These individual formulation types are known in principle and described, for example, in: Winnacker-Küchler, “Chemische Technologie” [Chemical Engineering], Volume 7, C. HauserVerlag Munich, 4th Edition 1986; van Falkenberg, “Pesticides Formulations”, Marcel Dekker N.Y., 2nd Ed. 1972-73; K. Martens, “Spray Drying Handbook”, 3rd Ed. 1979, G. Goodwin Ltd. London.

The formulation auxiliaries required, i.e. carriers and/or surface-active substances, such as inert materials, surfactants, solvents and further additives, are also known and described, for example, in: Watkins, “Handbook of Insecticide Dust Diluents and Carriers”, 2nd Ed., Darland Books, Caldwell N.J.; H. v. Olphen, “Introduction to Clay CoIloid Chemistry”, 2nd Ed., J. Wiley & Sons, N.Y.; Marsden, “Solvents Guide”, 2nd Ed., Interscience, N.Y. 1950; McCutcheon's, “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflachenaktive Äthylenoxidaddukte” [Surface-active ethylene oxide adducts], Wiss. Verlagsgesell., Stuttgart 1967; Winnacker-Küchler, “Chemische Technologie”, Volume 7, C. Hauser Verlag Munich, 4th Edition 1986.

Based on these formulations, it is also possible to prepare combinations with other pesticidally active substances, fertilizers and/or growth regulators, for example in the form of a readymix or a tank mix. Wettable powders are preparations which are uniformly dispersible in water and which, besides the active substance, also comprise, in addition to a diluent or inert material, wetters, for example polyoxethylated alkylphenols, polyoxethylated fatty alcohols, alkylsulfonates or alkylphenolsulfonates, and dispersants, for example, sodium lignosulfonate, sodium 2,2′-dinaphthylmethane-6,6′-disulfonate.

Emulsifiable concentrates are prepared by dissolving the active substance in an organic solvent, for example butanol, cyclohexanone, dimethylformamide, xylene or else higher-boiling aromatics or hydrocarbons with addition of one or more emulsifiers. Emulsifiers which can be used are, for example: calcium salts of alkylarylsulfonic acids, such as calcium dodecylbenzenesulfonate, or nonionic emulsifiers, such as fatty acid polyglycol esters, alkylaryl polyglycol ethers, fatty alcohol polyglycol ethers, propylene oxidelethylene oxide condensates, alkyl polyethers, sorbitan fatty acid esters, polyoxyethylene sorbitan fatty acid esters or polyoxethylene sorbitol esters.

Dusts are obtained by grinding the active substance with finely divided solid materials, for example talc or natural clays, such as kaolin, bentonite or pyrophyllite, or diatomaceous earth. Granules can be produced either by spraying the active substance onto adsorptive, granulated inert material or by applying active substance concentrates onto the surface of carriers such as sand, kaolinites or of granulated inert material, by means of binders, for example polyvinyl alcohol, sodium polyacrylate or alternatively mineral oils. Suitable active substances can also be granulated in the manner which is conventional for the production of fertilizer granules, if desired in a mixture with fertilizers.

The active substance concentration in wettable powders is usually about 10 to 90% by weight; the remaining 100% by weight is composed of conventional formulation components. In the case of emulsifiable concentrates, the active substance concentration can be approximately 5 to 80% by weight. Formulations in the form of dusts usually comprise 5 to 20% by weight of active substance, sprayable solutions approximately 2 to 20% by weight. In the case of granules, the active substance content depends partly on whether the active compound is in liquid or solid form and on which granulation auxiliaries, fillers etc. are being used.

In addition, the active substance formulations mentioned comprise, if appropriate, the adhesives, wetters, dispersants, emulsifiers, penetrants, solvents, fillers or carriers which are conventional in each case.

For use, the concentrates which are present in commercially available form are diluted, if appropriate, in the customary manner, for example using water in the case of wettable powders, emulsifiable concentrates, dispersions and in some cases also microgranules. Preparations in the form of dusts and granulated preparations and sprayable solutions are conventionally not further diluted with other inert materials prior to use.

The application rate required varies with the external conditions such as temperature, humidity and the like. It can vary within wide limits, for example between 0.0005 and 10.0 kg/ha or more of active substance, but it is preferably between 0.001 and 5 kg/ha active substance.

The active substances according to the invention may exist, in their commercially available formulations and in the use forms prepared with these formulations as mixtures with other active substances such as insecticides, attractants, sterilants, acaricides, nematicides, fungicides, growth regulators or herbicides.

The pesticides include, for example, phosphoric esters, carbamates, carboxylates, formamidines, tin compounds and substances produced by microorganisms.

Preferred components in mixtures are

1. From the Group of the Phosphorus Compounds

acephate, azamethiphos, azinphos-ethyl, azinphos-methyl, bromophos, bromophos-ethyl, cadusafos (F-67825), chlorethoxyphos, chlorfenvinphos, chlormephos, chlorpyrifos, chlorpyrifos-methyl, demeton, demeton-S-methyl, demeton-S-methyl sulfone, dialifos, diazinon, dichlorvos, dicrotophos, dimethoate, disulfoton, EPN, ethion, ethoprophos, etrimfos, famphur, fenamiphos, fenitriothion, fensulfothion, fenthion, fonofos, formothion, fosthiazate (ASC-66824) heptenophos, isazophos, isothioate, isoxathion, malathion, methacrifos, methamidophos, methidathion, salithion, mevinphos, monocrotophos, naled, omethoate, oxydemeton-methyl, parathion, parathion-methyl, phenthoate, phorate, phosalone, phosfolan, phosphocarb (BAS-301), phosmet, phosphamidon, phoxim, pirimiphos, pirimiphos-ethyl, pirimiphos-methyl, profenofos, propaphos, proetamphos, prothiofos, pyraclofos, pyridapenthion, quinalphos, sulprofos, temephos, terbufos, tebupirimfos, tetrachlorvinphos, thiometon, triazophos, trichlorphon, vamidothion;

2. From the Group of the Carbamates

alanycarb (OK-135), aldicarb, 2-sec-butyl phenylmethyl carbamate (BPMC), carbaryl, carbofuran, carbosulfan, cloethocarb, benfuracarb, ethiofencarb, furathiocarb, HCN-801, isoprocarb, methomyl, 5-methyl m-cumenylbutyryl(methyl)carbamate, oxamyl, pirimicarb, propoxur, thiodicarb, thiofanox, 1-methylthio(ethylideneamino)-N-methyl-N-(morpholinothio)carbamate (UC 51717), triazamate;

3. From the Group of the Carboxylic Esters

acrinathrin, allethrin, alphametrin, 5-benzyl-3-furylmethyl (E)-(1R)-cis-2,2-di-methyl-3-(2-oxothiolan-3-ylidenemethyl)cyclopropanecarboxylate, beta-cyfluthrin, beta-cypermethrin, bioallethrin, bioallethrin ((S)-cyclopentyl isomer), bioresmethrin, bifenthrin, (RS)-1-cyano-1-(6-phenoxy-2-pyridyl)methyl (1RS)-trans-3-(4-tert-butylphenyl)-2,2-dimethylcyclopropanecarboxylate (NCI 85193), cycloprothrin, cyfluthrin, cyhalothrin, cythithrin, cypermethrin, cyphenothrin, deltamethrin, empenthrin, esfenvalerate, fenfluthrin, fenpropathrin, fenvalerate, flucythrinate, flumethrin, fluvalinate (D isomer), imiprothrin (S-41311), lambda-cyhalothrin, permethrin, phenothrin ((R) isomer), prallethrin, pyrethrins (natural products), resmethrin, tefluthrin, tetramethrin, theta-cypermethrin (TD-2344), tralomethrin, transfluthrin, zeta-cypermethrin (F-56701);

4. From the Group of the Amidines

amitraz, chlorodimeform;

5. From the Group of the Tin Compounds

cyhexatin, fenbutatin oxide;

6. Others

abamectin, ABG-9008, acetamiprid, Anagrapha falcitera, AKD-1022, AKD-3059, ANS-118, Bacillus thuringiensis, Beauveria bassiana, bensultap, bifenazate (D-2341), binapacryl, BJL-932, bromopropylate, BTG-504, BTG-505, buprofezin, camphechlor, cartap, chlorbenzilate, chlorfenapyr, chlorfluazuron, 2-(4-chloro-phenyl)4,5-diphenylthiophene (UBI-T 930), chlorfentezine, chromafenozide (ANS-118), CG-216, CG-217, CG-234, A-184699, (2-naphthylmethyl)cyclopropane-carboxylate (Ro12-0470), cyromazin, diacloden (thiamethoxam), diafenthiuron, ethyl N-(3,5-dichloro-4-(1,1,2,3,3,3-hexafluoro-1-propyloxy)phenyl)carbamoyl)-2-chlorobenzocarboximidate, DDT, dicofol, diflubenzuron, N-(2,3-dihydro-3-methyl-1,3-thiazol-2-ylidene)-2,4-xylidine, dinobuton, dinocap, diofenolan, DPX-062, emamectin benzoate (MK-244), endosulfan, ethiprole (sulfethiprole), ethofenprox, etoxazole (YI-5301), fenazaquin, fenoxycarb, fipronil, fluazuron, flumite (Flufenzine, SZI-121), 2-fluoro-5-(4-(4-ethoxyphenyl)4-methyl-1-pentyl)diphenyl ether (MTI 800), granulosis and nuclear polyhedrosis viruses, fenpyroximate, fenthiocarb, flubenzimine, flucycloxuron, flufenoxuron, flufenprox (ICI-A5683), fluproxyfen, gamma-HCH, halofenozide (RH-0345), halofenprox (MTI-732), hexaflumuron (DE_{—}473), hexythiazox, HOI-9004, hydramethylnon (AC 217300), lufenuron, imidacloprid, indoxacarb (DPX-MP062), kanemite (AKD-2023), M-020, MTI-446, ivermectin, M-020, methoxyfenozide (intrepid, RH-2485), milbemectin, NC-1 96, neemgard, nitenpyram (TI-304), 2-nitromethyl-4,5-dihydro-6H-thiazine (DS 52618), 2-nitromethyl-3,4-dihydrothiazole (SD 35651), 2-nitromethylene-1,2-thiazinan-3-ylcarbamaldehyde (WL 108477), pyriproxyfen (S-71639), NC-196, NC-1111, NNI-9768, novaluron (MCW-275), OK-9701, OK-9601, OK-9602, propargite, pymethrozine, pyridaben, pyrimidifen (SU-8801), RH-0345, RH-2485, RYI-210, S-1283, S-1833, SB7242, SI-8601, silafluofen, silomadine (CG-177), spinosad, SU-9118, tebufenozide, tebufenpyrad (MK-239), teflubenzuron, tetradifon, tetrasul, thiacloprid, thiocyclam, TI-435, tolfenpyrad (OMI-88), triazamate (RH-7988), triflumuron, verbutin, vertalec (Mykotal), YI-5301.

The abovementioned components constitute known active substances, many of which are described in Ch. R. Worthing, S. B. Walker, The Pesticide Manual, 11th Edition, British Crop Protection Council, Farnham, 1997.

The active substance content of the use forms prepared from the commercially available formulations can be from 0.00000001 up to 95% by weight of acitve substance, preferably between 0.00001 and 1% by weight.

They are applied in a customary manner adapted to suit the use forms.

The active substances according to the invention are also suitable for controlling endo- and ectoparasites in the field of veterinary medicine or in the field of animal keeping.

Here, the active substances according to the invention are applied in a known manner, such as by oral administration in the form of, for example, tablets, capsules, drinks, granules, by dermal application in the form of, for example, dipping, spraying, pouring-on and spotting-on and dusting, and by parenteral administration, for example in the form of an injection.

Accordingly, the compounds of the formula (I) according to the invention can also be employed particularly advantageously in livestock keeping (for example cattle, sheep, pigs and poultry such as chickens, geese, etc.). In a preferred embodiment of the invention, the compounds are administered to the animals orally, if appropriate in suitable formulations and if appropriate together with the drinking water or the feed. Since excretion with the feces is highly efficient, this allows the development of insects in the animals' feces to be prevented in a very simple fashion. The dosages and formulations which are suitable in each case depend in particular on the species and developmental stage of the livestock and also on the risk of infection and can readily be determined and specified by the customary methods. For example, the compounds can be employed in cattle in dosages of 0.01 to 1 mg/kg bodyweight.

Besides the applications methods mentioned so far, the active substances of the formula (I) according to the invention show an outstanding systemic action. The active substances can therefore also be introduced into the plants via subterranean or aerial plant organs (roots, stalks, leaves) when the active substances are applied to the immediate environment of the plant either in liquid or in solid form (for example granules in the case of soil application, application into paddy fields).

In addition, the active substances according the invention can be employed in a particular manner for the treatment of vegetative and generative propagation material, such as, for example, seed of, for example, cereals, vegetables, cotton, rice, sugarbeet and other crop plants and ornamentals, of bulbs, cuttings and tubers of other crop plants and ornamentals which are propagated by vegetative propagation. To this end, treatment may take place prior to sowing or the planting procedure (for example by specific seed-coating techniques, by dressing in liquid or solid form, or by the seed box treatment), during the sowing procedure or during planting, or after the sowing or planting procedure, using specific application techniques (for example row treatment). The active substance quantity applied may vary within a substantial range, depending on the purpose. In general, the application rates are between 1 g and 10 kg of active substance per hectare of soil surface.

The compounds of the formula (I) can also be employed for controlling harmful plants in crops of known genetically modified plants, or genetically modified plants yet to be developed. As a rule, the transgenic plants are distinguished by particular, advantageous properties, for example by resistances to certain crop protection agents, resistances to plant diseases or to pathogens of plant diseases such as certain insects or microorganisms such as fungi, bacteria or viruses. Other particular properties relate to for example the harvested material with regard to quantity, quality, shelf-life, composition and specific constituents. For example, transgenic plants with an increased starch content or with an altered starch quality or those whose harvested material has a different fatty acid spectrum are known.

Preferred is the use in economically important transgenic crops of useful plants and ornamentals, for example of cereals such as wheat, barley, rye, oats, sorghum and millet, rice, cassava and maize, or else crops of sugarbeet, cotton, soya, oilseed rape, potatoes, tomatoes, peas and other vegetables.

When used in transgenic cultures, in particular those with resistances to insects, effects are frequently observed—in addition to the effects against harmful organisms which can be observed in other crops—which are specific for application in the particular transgenic crop, for example an altered, or specifically widened, spectrum of pests which can be controlled, or altered application rates which can be employed for application.

The invention therefore also relates to the use of compounds of the formula (I) for controlling harmful organisms in transgenic crop plants.

In addition to the direct application to the pests, application of the compounds according to the invention comprises any other application where compounds of the formula (I) act on the pests. Such indirect applications can be, for example, the use of compounds which break down, or are broken down, to give compounds of the formula (I), for example in the soil, the plants or the pests.

In addition to their lethal effect on pests, the compounds of the formula (I) are also distinguished by a pronounced repellant effect.

A repellant for the purposes of the invention is a substance or substance mixture which has a warding-off or fending-off effect on other live organisms, in particular harmful pests and nuisance pests. The term also encompasses effects such as the antifeeding effect, where the intake of feed is disturbed or prevented (antifeedant effect), supression of oviposition, or an effect on the development of the population.

The invention therefore also relates to the use of compounds of the formula (I) for achieving the abovementioned effects, in particular in the case of the pests stated in the biological examples.

The invention also relates to a method of fending off, or warding off, harmful organisms, where one or more compounds of the formula (I) are applied to the site from which the harmful organisms are to be fended off or warded off.

In the case of a plant, application may mean, for example, a treatment of the plant, but also of the seed.

As regards the effect on populations, it is interesting to note that effects can also be observed in succession during the development of a population, where summation may take place. In such a case, the individual effect itself may only have an efficacy of markedly less than 100% but in total an efficacy of 100% is still achieved in the end.

Moreover, the compounds of the formula (I) are distinguished by the fact that the composition is usually applied earlier than in the case of a direct control, if the abovementioned effects are to be exploited. The effect frequently lasts over a long period, so that a duration of action of over 2 months is achieved.

The effects are not only found in insects, but also in spider mites and molluscs.

The examples which follow are intended to illustrate the invention.

### A. CHEMICAL EXAMPLES

### Example a

### Table 1, Example 15

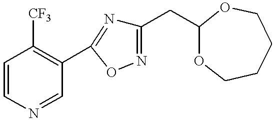

3-(1,3-Dioxepan-2-ylmethyl)-5-(4-trifluoromethyl-3-pyridyl)-1,2,4-oxadiazole

1.4g (4 mmol) of (1,3-dioxepan-2-yl) O-(4-trifluoromethyinicotinoyl)acetamide oxime were dissolved in 25 ml of tetrahydrofuran and, after addition of 0.45 g (4 mmol) of potassium tert-butoxide, stirred for 6 hours at 50° C. After cooling, the mixture was concentrated and the residue was taken up in water/dichloromethane. The residue which remained after concentration of the organic phase was purified by chromatography on silica gel (heptane/ethyl acetate 1:1). This gave 0.66 g (50.1% of theory) of product as colorless oil.

Preparation of the precursors

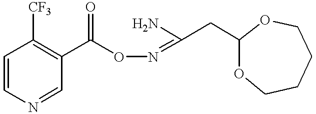

(1,3-Dioxepan-2-yl) O-(4-trifluoromethylnicotinoyl)acetamide Oxime

10.1 g (53 mmol) of 4-trifluoromethylnicotinic acid and 8.6 g (53 mmol) of carbonyidiimidazole were stirred in 150 ml of tetrahydrofuran at 50° C. until the evolution of gas had ceased. 10.0 g (58 mmol) of 2-(1,3-dioxepan-2-yl)acetamide oxime were subsequently added and stirring was continued for 6 hours at 50° C. For work-up, the mixture was concentrated and the residue was taken up in dichloromethane and water. The organic phase was again washed with water, dried and concentrated. For purification, the semi-solid residue was suspended in ethyl acetate and the suspension was filtered off with suction. This gave 5.7 g of product as colorless crystals. Column filtration of the ethyl acetate filtrate on silica gel (ethyl acetate) gave a further 3.2 g of product. Total yield 8.9 g (48.4% of theory). m.p. 100-102° C.

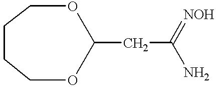

(1,3-Dioxepan-2-yl)acetamide Oxime

A solution of 10.9 g (77 mmol) of (1,3-dioxepan-2-yl)acetonitrile and 6.4 g (92 mmol) of hydroxylamine hydrochloride in 100 ml of methanol was treated with 16.6 g (92 mmol) of 30% strength methanolic sodium methoxide solution and then stirred first for 4 hours at room temperature and then for 5 hours at 50° C. After cooling, sodium chloride was removed by filtration and the filtrate was concentrated.

Following column filtration of the residue on silica gel (ethyl acetate/methanol 4:1), 10.5 g (78.3% of theory) of product were obtained as colorless oil.

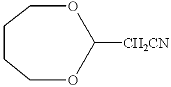

(1,3-Dioxepan-2-yl)acetonitrile

34.5 g (0.3 mol) of 3,3-dimethoxypropionitrile and 54.1 g (0.6 mol) of 1,4-butanediol were heated to 100° C. together with 0.5 g of p-toluenesulfonic acid monohydrate in 200 ml of toluene, and methanol which formed was distilled off.

For work-up, the mixture was twice extracted by stirring with dilute sodium hydroxide solution and with water and the organic phase was dried and concentrated. Following vacuum distillation, 11.1 g (26.0% of theory) of product were obtained as colorless oil.

b.p. at 12 mm: 104-106° C.

### Example b

### Table 1, Example 8

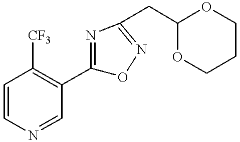

3-(1,3-Dioxan-2-ylmethyl)-5-(4-trifluoromethyl-3-pyridyl)-1,2,4-oxadiazole.

910 mg (3 mmol) of 3-(2,2-dimethoxyethyl)-5-(4-trifluoromethyl)-1,2,4-oxadiazole (WO-A-98/57969, Ex. 376) together with 100 mg of p-toluenesulfonic acid monohydrate and 15 ml of toluene were heated for 4 hours at 100° C. After cooling, the mixture was twice extracted by stirring with dilute sodium hydroxide solution and then with water. The organic phase was dried and concentrated. This gave 830 mg (87.8% of theory) of product as pale yellow oil.

TABLE I

| Ex. | | | | | m.p | |
|---|---|---|---|---|---|---|
| No. | A | n | X | Het | [° C.] | Note |
| 1 | CH | 0 | CH₂ | 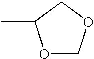 | oil | |
| 2 | N | 0 | CH₂ | ″ | | |
| 3 | CH | 1 | CH₂ | ″ | | |
| 4 | CH | 0 | (CH₂)₂ | ″ | oil | |
| 5 | CH | 0 | (CH₂)₃ | ″ | | |
| 6 | CH | 0 | CH₂CH(CH₃) | ″ | | |
| 7 | CH | 0 | CH₂(CH) | ″ | | |
| | | | (CH₃)CH₂ | | | |
| 8 | CH | 0 | CH₂ | 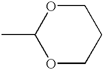 | oil | |
| 9 | N | 0 | CH₂ | ″ | | |
| 10 | CH | 1 | CH₂ | ″ | | |
| 11 | CH | 0 | (CH₂)₂ | ″ | oil | |
| 12 | CH | 0 | (CH₂)₃ | ″ | | |
| 13 | CH | 0 | CH₂CH(CH₃) | ″ | | |
| 14 | CH | 0 | CH₂CH(CH₃)CH₂ | ″ | | |
| 15 | CH | 0 | CH₂ | 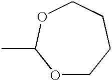 | oil | |
| 16 | N | 0 | CH₂ | ″ | | |
| 17 | CH | 0 | (CH₂)₂ | ″ | oil | |
| 18 | CH | 0 | CH₂ | 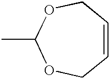 | | |
| 19 | CH | 0 | (CH₂)₂ | ″ | oil | |
| 20 | CH | 0 | CH₂ | 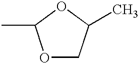 | oil | cis/trans mixture |
| 21 | CH | 0 | (CH₂)₂ | ″ | | |
| 22 | CH | 0 | CH₂ | 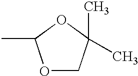 | | |
| 23 | CH | 0 | (CH₂)₂ | ″ | | |
| 24 | CH | 0 | CH₂ | 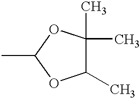 | oil | cis/trans mixture |
| 25 | CH | 0 | (CH₂)₂ | ″ | | |
| 26 | CH | 0 | CH₂ | 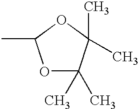 | | |
| 27 | CH | 0 | CH₂ | ″ | | |
| 28 | CH | 0 | CH₂ | 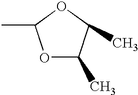 | oil | cis/trans mixture |
| 29 | CH | 0 | (CH₂)₂ | ″ | | |
| 30 | CH | 0 | CH₂ | 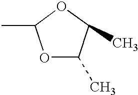 | oil | |
| 31 | CH | 0 | (CH₂)₂ | ″ | | |
| 32 | CH | 0 | CH₂ | 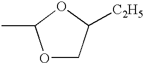 | | |
| 0 | | | | | | |
| 33 | CH | 0 | (CH₂)₂ | ″ | | |
| 34 | CH | 0 | CH₂ | 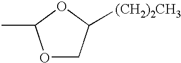 | oil | cis/trans mixture |
| 35 | CH | 0 | CH₂ | ″ | | |
| 36 | CH | 0 | CH₂ | 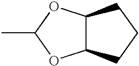 | oil | |
| 37 | CH | 0 | (CH₂)₂ | ″ | oil | |
| 38 | CH | 0 | CH₂ | 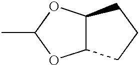 | | |
| 39 | CH | 0 | (CH₂)₂ | ″ | | |
| 40 | CH | 0 | CH₂ | 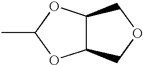 | oil | |
| 41 | CH | 0 | (CH₂)₂ | ″ | oil | cis/trans mixture |
| 42 | CH | 0 | CH₂ | 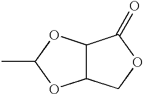 | | |
| 43 | CH | 0 | (CH₂)₂ | ″ | | |
| 44 | CH | 0 | CH₂ | 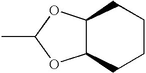 | | |
| 45 | CH | 0 | (CH₂)₂ | ″ | | |
| 46 | CH | 0 | CH₂ | 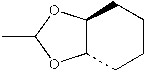 | | |
| 47 | CH | 0 | (CH₂)₂ | ″ | | |
| 48 | CH | 0 | CH₂ | 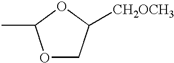 | | |
| 49 | CH | 0 | (CH₂)₂ | ″ | | |
| 50 | CH | 0 | CH₂ | 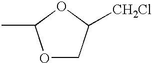 | | |
| 0 | | | | | | |
| 51 | CH | 0 | (CH₂)₂ | ″ | | |
| 52 | CH | 0 | CH₂ | 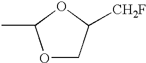 | oil | |
| 53 | CH | 0 | (CH₂)₂ | ″ | | |
| 54 | CH | 0 | CH₂ | 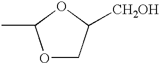 | oil | cis/trans mixture |
| 55 | CH | 0 | (CH₂)₂ | ″ | | |
| 56 | CH | 0 | CH₂ | 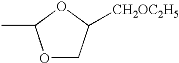 | oil | cis/trans mixture |
| 57 | CH | 0 | (CH₂)₂ | ″ | | |
| 58 | CH | 0 | CH₂ | 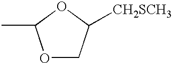 | oil | cis/trans mixture |
| 59 | CH | 0 | (CH₂)₂ | ″ | | |
| 60 | CH | 0 | CH₂ | 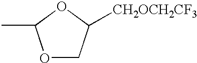 | | |
| 61 | CH | 0 | (CH₂)₂ | ″ | | |
| 62 | CH | 0 | CH₂ | 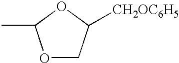 | | |
| 63 | CH | 0 | (CH₂)₂ | ″ | | |
| 64 | CH | 0 | CH₂ | 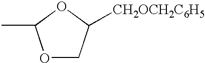 | | |
| 65 | CH | 0 | (CH₂)₂ | ″ | | |
| 66 | CH | 0 | CH₂ | 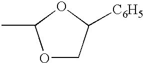 | oil | |
| 67 | CH | 0 | (CH₂)₂ | ″ | | |
| 68 | CH | 0 | CH₂ | 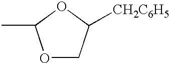 | | |
| 69 | CH | 0 | (CH₂)₂ | ″ | | |
| 70 | CH | 0 | CH₂ | 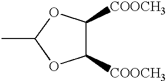 | | |
| 71 | CH | 0 | (CH₂)₂ | ″ | | |
| 72 | CH | 0 | CH₂ | 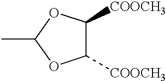 | | |
| 73 | CH | 0 | (CH₂)₂ | ″ | | |
| 74 | CH | 0 | CH₂ | 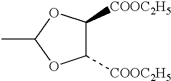 | | |
| 75 | CH | 0 | (CH₂)₂ | ″ | | |
| 76 | CH | 0 | CH₂ | 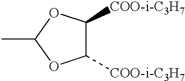 | | |
| 77 | CH | 0 | (CH₂)₂ | ″ | | |
| 78 | CH | 0 | CH₂ | 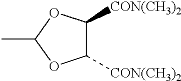 | | |
| 79 | CH | 0 | (CH₂)₂ | ″ | | |
| 80 | CH | 0 | CH₂ | 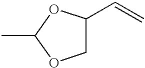 | | |
| 81 | CH | 0 | (CH₂)₂ | ″ | | |
| 82 | CH | 0 | CH₂ | 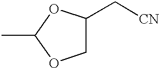 | | |
| 83 | CH | 0 | (CH₂)₂ | ″ | | |
| 84 | CH | 0 | CH₂ | 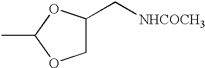 | | |
| 85 | CH | 0 | (CH₂)₂ | ″ | | |
| 86 | CH | 0 | CH₂ | 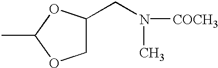 | | |
| 87 | CH | 0 | (CH₂)₂ | ″ | | |
| 88 | CH | 0 | CH₂ | 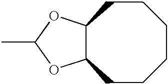 | | |
| 89 | CH | 0 | (CH₂)₂ | ″ | | |
| 90 | CH | 0 | CH₂ | 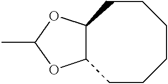 | | |
| 91 | CH | 0 | (CH₂)₂ | ″ | | |
| 92 | CH | 0 | CH₂ | 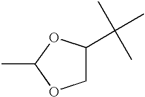 | | |
| 93 | CH | 0 | (CH₂)₂ | ″ | | |
| 94 | CH | 0 | CH₂ | 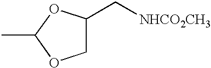 | | |
| 95 | CH | 0 | (CH₂)₂ | ″ | | |
| 96 | CH | 0 | CH₂ | 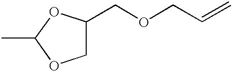 | | |
| 97 | CH | 0 | (CH₂)₂ | ″ | | |
| 98 | CH | 0 | CH₂ | 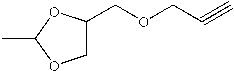 | | |
| 99 | CH | 0 | (CH₂)₂ | ″ | | |
| 100 | CH | 0 | CH₂ | 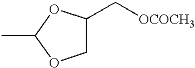 | | |
| 101 | CH | 0 | (CH₂)₂ | ″ | | |
| 102 | CH | 0 | CH₂ | 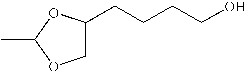 | | |
| 103 | CH | 0 | (CH₂)₂ | ″ | | |
| 104 | CH | 0 | CH₂ | 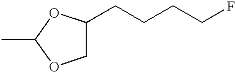 | | |
| 105 | CH | 0 | (CH₂)₂ | ″ | | |
| 106 | CH | 0 | CH₂ | 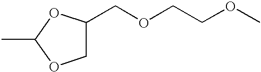 | | |
| 107 | CH | 0 | (CH₂)₂ | ″ | | |
| 108 | CH | 0 | CH₂ | 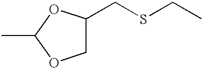 | | |
| 109 | CH | 0 | (CH₂)₂ | ″ | | |
| 110 | CH | 0 | CH₂ | 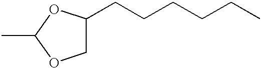 | | |
| 111 | CH | 0 | (CH₂)₂ | ″ | | |
| 112 | CH | 0 | CH₂ | 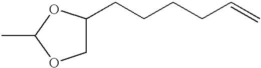 | | |
| 113 | CH | 0 | (CH₂)₂ | ″ | | |
| 114 | CH | 0 | CH₂ | 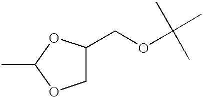 | | |
| 115 | CH | 0 | (CH₂)₂ | ″ | | |
| 116 | CH | 0 | CH₂ | 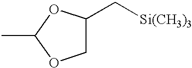 | | |
| 117 | CH | 0 | (CH₂)₂ | ″ | | |
| 118 | CH | 0 | CH₂ | 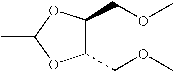 | oil | |
| 119 | CH | 0 | (CH₂)₂ | ″ | | |
| 120 | CH | 0 | CH₂ | 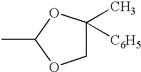 | | |
| 121 | CH | 0 | (CH₂)₂ | ″ | | |
| 122 | CH | 0 | CH₂ | 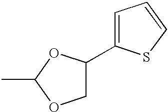 | | |
| 123 | CH | 0 | (CH₂)₂ | ″ | | |
| 124 | CH | 0 | CH₂ | 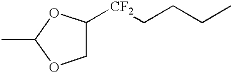 | | |
| 125 | CH | 0 | (CH₂)₂ | ″ | | |
| 126 | CH | 0 | CH₂ | 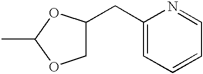 | | |
| 127 | CH | 0 | (CH₂)₂ | ″ | | |
| 128 | CH | 0 | CH₂ | 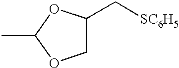 | | |
| 129 | CH | 0 | (CH₂)₂ | ″ | | |
| 130 | CH | 0 | CH₂ | 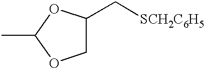 | | |
| 131 | CH | 0 | (CH₂)₂ | ″ | | |
| 132 | CH | 0 | CH₂ | 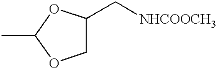 | | |
| 133 | CH | 0 | (CH₂)₂ | ″ | | |
| 134 | CH | 0 | CH₂ | 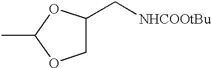 | | |
| 135 | CH | 0 | (CH₂)₂ | ″ | | |
| 136 | CH | 0 | CH₂ | 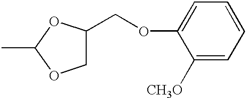 | | |
| 137 | CH | 0 | (CH₂)₂ | ″ | | |
| 138 | CH | 0 | CH₂ | 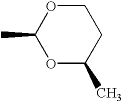 | oil | |
| 139 | CH | 0 | (CH₂)₂ | ″ | | |
| 140 | CH | 0 | CH₂ | 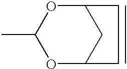 | | |
| 141 | CH | 0 | (CH₂)₂ | ″ | | |
| 142 | CH | 0 | CH₂ | 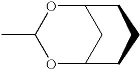 | | |
| 143 | CH | 0 | (CH₂)₂ | ″ | | |
| 144 | CH | 0 | CH₂ | 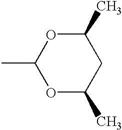 | | |
| 145 | CH | 0 | (CH₂)₂ | ″ | oil | |
| 146 | CH | 0 | CH₂ | 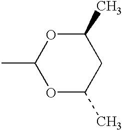 | oil | |
| 147 | CH | 0 | (CH₂)₂ | ″ | | |
| 148 | CH | 0 | CH₂ | 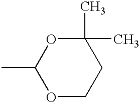 | oil | |
| 149 | CH | 0 | (CH₂)₂ | ″ | | |
| 150 | CH | 0 | CH₂ | 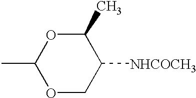 | | |
| 151 | CH | 0 | (CH₂)₂ | ″ | | |
| 152 | CH | 0 | CH₂ | 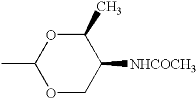 | | |
| 153 | CH | 0 | (CH₂)₂ | ″ | | |
| 154 | CH | 0 | CH₂ | 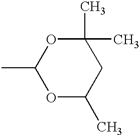 | oil | |
| 155 | CH | 0 | (CH₂)₂ | ″ | | |
| 156 | CH | 0 | CH₂ | 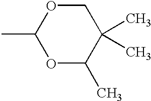 | | |
| 157 | CH | 0 | (CH₂)₂ | ″ | | |
| 158 | CH | 0 | CH₂ | 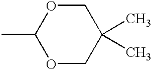 | oil | |
| 159 | CH | 0 | (CH₂)₂ | ″ | oil | |
| 160 | CH | 0 | CH₂ | 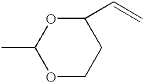 | | |
| 161 | CH | 0 | (CH₂)₂ | ″ | | |
| 162 | CH | 0 | CH₂ | 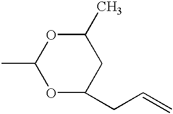 | | |
| 163 | CH | 0 | (CH₂)₂ | ″ | | |
| 164 | CH | 0 | CH₂ | 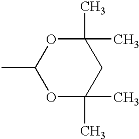 | | |
| 165 | CH | 0 | (CH₂)₂ | ″ | | |
| 166 | CH | 0 | CH₂ | 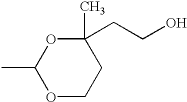 | | |
| 167 | CH | 0 | (CH₂)₂ | ″ | | |
| 168 | CH | 0 | CH₂ | 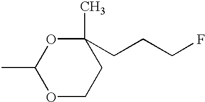 | | |
| 169 | CH | 0 | (CH₂)₂ | ″ | | |
| 170 | CH | 0 | CH₂ | 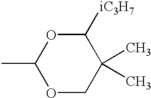 | | |
| 171 | CH | 0 | (CH₂)₂ | ″ | | |
| 172 | CH | 0 | CH₂ | 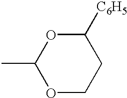 | | |
| 173 | CH | 0 | (CH₂)₂ | ″ | | |
| 174 | CH | 0 | CH₂ | 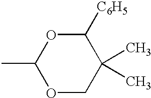 | | |
| 175 | CH | 0 | (CH₂)₂ | ″ | | |
| 176 | CH | 0 | CH₂ | 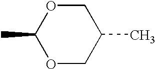 | oil | |
| 177 | CH | 0 | (CH₂)₂ | ″ | | |
| 178 | CH | 0 | CH₂ | 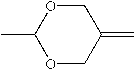 | | |
| 179 | CH | 0 | (CH₂)₂ | ″ | oil | |
| 180 | CH | 0 | CH₂ | 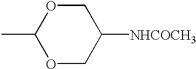 | | |
| 181 | CH | 0 | (CH₂)₂ | ″ | | |
| 182 | CH | 0 | CH₂ | 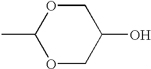 | | |
| 183 | CH | 0 | (CH₂)₂ | ″ | | |
| 184 | CH | 0 | CH₂ | 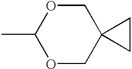 | 104-105 | |
| 185 | CH | 0 | (CH₂)₂ | ″ | | |
| 186 | CH | 0 | CH₂ | 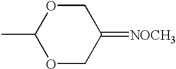 | | |
| 187 | CH | 0 | (CH₂)₂ | 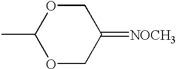 | | |
| 188 | CH | 0 | CH₂ | 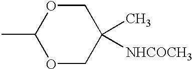 | | |
| 189 | CH | 0 | (CH₂)₂ | ″ | | |
| 190 | CH | 0 | CH₂ | 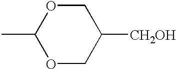 | | |
| 191 | CH | 0 | (CH₂)₂ | ″ | | |
| 192 | CH | 0 | CH₂ | 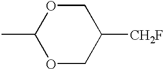 | | |
| 193 | CH | 0 | (CH₂)₂ | ″ | | |
| 194 | CH | 0 | CH₂ | 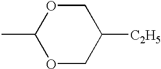 | | |
| 195 | CH | 0 | (CH₂)₂ | ″ | | |
| 196 | CH | 0 | CH₂ | 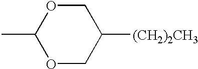 | | |
| 197 | CH | 0 | (CH₂)₂ | ″ | | |
| 198 | CH | 0 | CH₂ | 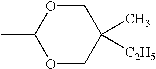 | | |
| 199 | CH | 0 | (CH₂)₂ | ″ | | |
| 200 | CH | 0 | CH₂ | 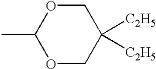 | | |
| 201 | CH | 0 | (CH₂)₂ | ″ | | |
| 202 | CH | 0 | CH₂ | 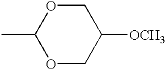 | | |
| 203 | CH | 0 | (CH₂)₂ | ″ | | |
| 204 | CH | 0 | CH₂ | 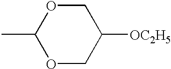 | | |
| 205 | CH | 0 | (CH₂)₂ | ″ | | |
| 206 | CH | 0 | CH₂ | 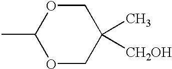 | | |
| 207 | CH | 0 | (CH₂)₂ | ″ | | |
| 208 | CH | 0 | CH₂ | 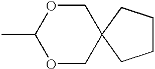 | | |
| 209 | CH | 0 | (CH₂)₂ | ″ | | |
| 210 | CH | 0 | CH₂ | 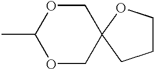 | | |
| 211 | CH | 0 | (CH₂)₂ | ″ | | |
| 212 | CH | 0 | CH₂ | 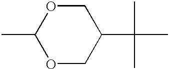 | | |
| 213 | CH | 0 | (CH₂)₂ | ″ | | |
| 214 | CH | 0 | CH₂ | 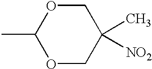 | | |
| 215 | CH | 0 | (CH₂)₂ | ″ | oil | isomer mixture |
| 216 | CH | 0 | CH₂ | 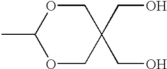 | | |
| 217 | CH | 0 | (CH₂)₂ | ″ | | |
| 218 | CH | 0 | CH₂ | 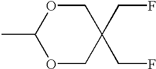 | | |
| 219 | CH | 0 | (CH₂)₂ | ″ | | |
| 220 | CH | 0 | CH₂ | 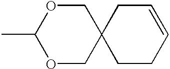 | | |
| 221 | CH | 0 | (CH₂)₂ | ″ | | |
| 222 | CH | 0 | CH₂ | 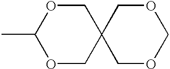 | | |
| 223 | CH | 0 | (CH₂)₂ | ″ | | |
| 224 | CH | 0 | CH₂ | 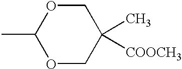 | | |
| 225 | CH | 0 | (CH₂)₂ | ″ | | |
| 226 | CH | 0 | CH₂ | 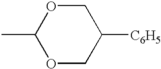 | | |
| 227 | CH | 0 | (CH₂)₂ | ″ | oil | |
| 228 | CH | 0 | CH₂ | 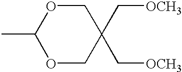 | | |
| 229 | CH | 0 | (CH₂)₂ | ″ | | |
| 230 | CH | 0 | CH₂ | 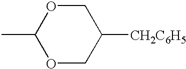 | | |
| 231 | CH | 0 | (CH₂)₂ | ″ | | |
| 232 | CH | 0 | CH₂ | 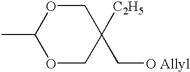 | | |
| 233 | CH | 0 | (CH₂)₂ | ″ | | |
| 234 | CH | 0 | CH₂ | 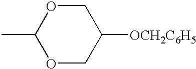 | | |
| 235 | CH | 0 | (CH₂)₂ | ″ | | |
| 236 | CH | 0 | CH₂ | 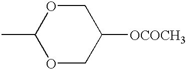 | | |
| 237 | CH | 0 | (CH₂)₂ | ″ | | |
| 238 | CH | 0 | CH₂ | 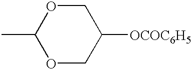 | | |
| 239 | CH | 0 | (CH₂)₂ | ″ | | |
| 240 | CH | 0 | CH₂ | 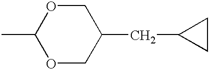 | | |
| 241 | CH | 0 | (CH₂)₂ | ″ | | |
| 242 | CH | 0 | CH₂ | 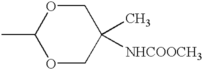 | | |
| 243 | CH | 0 | (CH₂)₂ | ″ | | |
| 244 | CH | 0 | CH₂ | 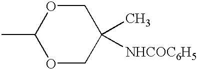 | | |
| 245 | CH | 0 | (CH₂)₂ | ″ | | |
| 246 | CH | 0 | CH₂ | 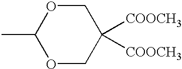 | | |
| 247 | CH | 0 | (CH₂)₂ | ″ | | |
| 248 | CH | 0 | CH₂ | 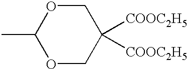 | | |
| 249 | CH | 0 | (CH₂)₂ | ″ | | |
| 250 | CH | 0 | CH₂ | 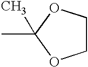 | | |
| 251 | CH | 0 | (CH₂)₂ | ″ | | |
| 252 | CH | 0 | CH₂ | 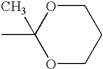 | | |
| 253 | CH | 0 | (CH₂)₂ | ″ | | |
| 254 | CH | 0 | CH₂ | 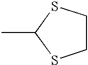 | oil | |
| 255 | CH | 0 | (CH₂)₂ | ″ | | |
| 256 | CH | 0 | CH₂ | 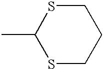 | | |
| 257 | CH | 0 | (CH₂)₂ | ″ | | |
| 258 | CH | 0 | CH₂ | 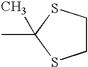 | | |
| 259 | CH | 0 | (CH₂)₂ | ″ | | |
| 260 | CH | 0 | CH₂ | 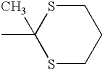 | | |
| 261 | CH | 0 | (CH₂)₂ | ″ | | |
| 262 | CH | 0 | CH₂ | 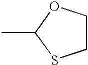 | | |
| 263 | CH | 0 | (CH₂)₂ | ″ | | |
| 264 | CH | 0 | CH₂ | 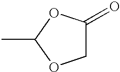 | | |
| 265 | CH | 0 | (CH₂)₂ | ″ | | |
| 266 | CH | 0 | CH₂ | 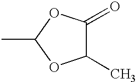 | | |
| 267 | CH | 0 | (CH₂)₂ | ″ | | |
| 268 | CH | 0 | CH₂ | 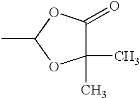 | | |
| 269 | CH | 0 | (CH₂)₂ | ″ | | |
| 270 | CH | 0 | CH₂ | 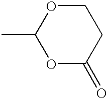 | | |
| 271 | CH | 0 | (CH₂)₂ | ″ | | |
| 272 | CH | 0 | CH₂ | 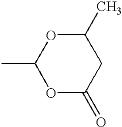 | | |
| 273 | CH | 0 | (CH₂)₂ | ″ | | |
| 274 | CH | 0 | CH₂ | 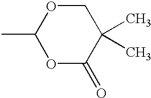 | | |
| 275 | CH | 0 | (CH₂)₂ | ″ | | |
| 276 | CH | 0 | CH₂ | 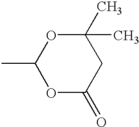 | | |
| 277 | CH | 0 | (CH₂)₂ | ″ | | |
| 278 | CH | 0 | CH₂ | 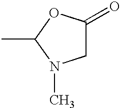 | | |
| 279 | CH | 0 | (CH₂)₂ | ″ | | |
| 280 | CH | 0 | CH₂ | 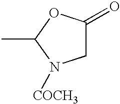 | | |
| 281 | CH | 0 | (CH₂)₂ | ″ | | |
| 282 | CH | 0 | CH₂ | 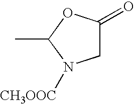 | | |
| 283 | CH | 0 | (CH₂)₂ | ″ | | |
| 284 | CH | 0 | CH₂ | 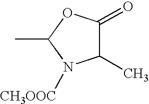 | | |
| 285 | CH | 0 | (CH₂)₂ | ″ | | |
| 286 | CH | 0 | CH₂ | 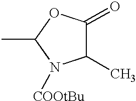 | | |
| 287 | CH | 0 | (CH₂)₂ | ″ | | |
| 288 | CH | 0 | CH₂ | 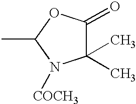 | | |
| 289 | CH | 0 | (CH₂)₂ | ″ | | |
| 290 | CH | 0 | CH₂ | 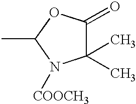 | | |
| 291 | CH | 0 | (CH₂)₂ | ″ | | |
| 292 | CH | 0 | CH₂ | 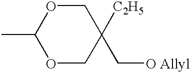 | | |
| 293 | CH | 0 | (CH₂)₂ | ″ | | |
| 294 | CH | 0 | CH₂ | 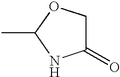 | | |
| 295 | CH | 0 | (CH₂)₂ | ″ | | |
| 296 | CH | 0 | CH₂ | 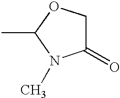 | | |
| 297 | CH | 0 | (CH₂)₂ | ″ | | |
| 298 | CH | 0 | CH₂ | 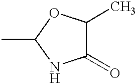 | | |
| 299 | CH | 0 | (CH₂)₂ | ″ | | |
| 300 | CH | 0 | CH₂ | 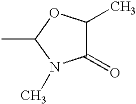 | | |
| 301 | CH | 0 | (CH₂)₂ | ″ | | |
| 301 | CH | 0 | CH₂ | 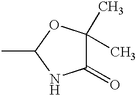 | | |
| 303 | CH | 0 | (CH₂)₂ | ″ | | |
| 304 | CH | 0 | CH₂ | 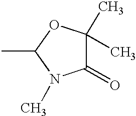 | | |
| 305 | CH | 0 | (CH₂)₂ | ″ | | |
| 306 | CH | 0 | CH₂ | 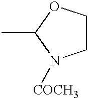 | | |
| 307 | CH | 0 | (CH₂)₂ | ″ | | |
| 308 | CH | 0 | CH₂ | 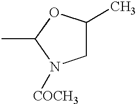 | | |
| 309 | CH | 0 | (CH₂)₂ | ″ | | |
| 310 | CH | 0 | CH₂ | 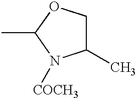 | | |
| 311 | CH | 0 | (CH₂)₂ | ″ | | |
| 312 | CH | 0 | CH₂ | 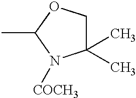 | | |
| 313 | CH | 0 | (CH₂)₂ | ″ | | |
| 314 | CH | 0 | CH₂ | 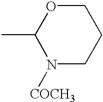 | | |
| 315 | CH | 0 | (CH₂)₂ | ″ | | |
| 316 | CH | 0 | CH₂ | 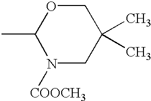 | | |
| 317 | CH | 0 | (CH₂)₂ | ″ | | |
| 318 | CH | 0 | CH₂ | 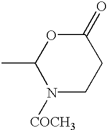 | | |
| 319 | CH | 0 | (CH₂)₂ | ″ | | |
| 320 | CH | 0 | CH₂ | 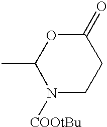 | | |
| 321 | CH | 0 | (CH₂)₂ | ″ | | |
| 322 | CH | 0 | CH₂ | 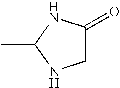 | | |
| 323 | CH | 0 | (CH₂)₂ | ″ | | |
| 324 | CH | 0 | CH₂ | 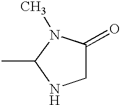 | | |
| 325 | CH | 0 | (CH₂)₂ | ″ | | |
| 326 | CH | 0 | CH₂ | 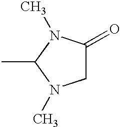 | | |
| 327 | CH | 0 | (CH₂)₂ | ″ | | |
| 328 | CH | 0 | CH₂ | 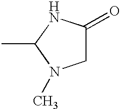 | | |
| 329 | CH | 0 | (CH₂)₂ | ″ | | |
| 330 | CH | 0 | CH₂ | 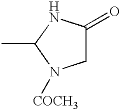 | | |
| 331 | CH | 0 | (CH₂)₂ | ″ | | |
| 332 | CH | 0 | CH₂ | 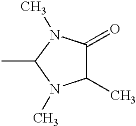 | | |
| 333 | CH | 0 | (CH₂)₂ | ″ | | |
| 334 | CH | 0 | CH₂ | 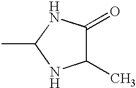 | | |
| 335 | CH | 0 | (CH₂)₂ | ″ | | |
| 336 | CH | 0 | CH₂ | 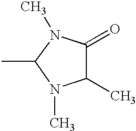 | | |
| 337 | CH | 0 | (CH₂)₂ | ″ | | |
| 338 | CH | 0 | CH₂ | 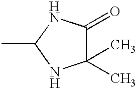 | | |
| 339 | CH | 0 | (CH₂)₂ | ″ | | |
| 340 | CH | 0 | CH₂ | 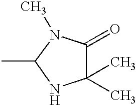 | | |
| 341 | CH | 0 | (CH₂)₂ | ″ | | |
| 342 | CH | 0 | CH₂ | 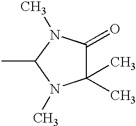 | | |
| 343 | CH | 0 | (CH₂)₂ | ″ | | |
| 344 | CH | 0 | CH₂ | 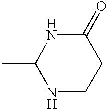 | | |
| 345 | CH | 0 | (CH₂)₂ | ″ | | |
| 346 | CH | 0 | CH₂ | 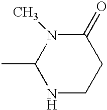 | | |
| 347 | CH | 0 | (CH₂)₂ | ″ | | |
| 348 | CH | 0 | CH₂ | 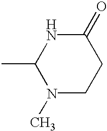 | | |
| 349 | CH | 0 | (CH₂)₂ | ″ | | |
| 350 | CH | 0 | CH₂ | 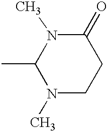 | | |
| 351 | CH | 0 | (CH₂)₂ | ″ | | |
| 352 | CH | 0 | CH₂ | 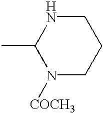 | | |
| 353 | CH | 0 | (CH₂)₂ | ″ | | |
| 354 | CH | 0 | CH₂ | 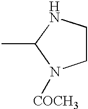 | | |
| 355 | CH | 0 | (CH₂)₂ | ″ | | |
| 356 | CH | 0 | CH₂ | 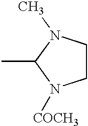 | | |
| 357 | CH | 0 | (CH₂)₂ | ″ | | |
| 358 | CH | | CH₂ | 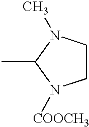 | | |
| 359 | CH | 0 | (CH₂)₂ | ″ | | |
| 360 | CH | 0 | CH₂ | 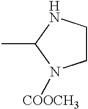 | | |
| 361 | CH | 0 | (CH₂)₂ | ″ | | |
| 362 | CH | 0 | CH₂ | 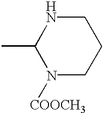 | | |
| 363 | CH | 0 | (CH₂)₂ | ″ | | |
| 364 | CH | 0 | CH₂ | 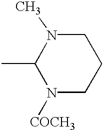 | | |
| 365 | CH | 0 | (CH₂)₂ | ″ | | |
| 366 | CH | 0 | CH₂ | 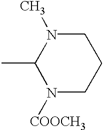 | | |
| 367 | CH | 0 | (CH₂)₂ | ″ | | |
| 368 | CH | 0 | — | 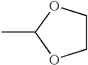 | oil | |
| 369 | CH | 0 | — | 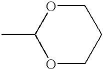 | oil | |
| 370 | CH | 0 | — | 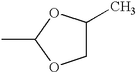 | oil | |
| 371 | CH | 0 | — | 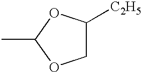 | oil | |
| 372 | CH | 0 | — | 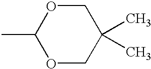 | oil | |
| 373 | CH | 0 | — | 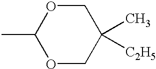 | oil | |
| 373-A | CH | 0 | CH₂ | 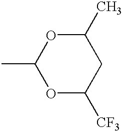 | oil | |
| 373-B | CH | 0 | CH₂ | 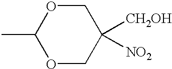 | 154-155 | |

TABLE 2

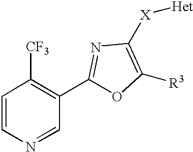

| Ex. No. | R³ | X | Het | m.p. [° C.] | Note |
|---|---|---|---|---|---|
| 374 | H | CH₂ | 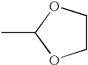 | | |
| 375 | H | (CH₂)₂ | 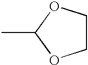 | | |
| 376 | CH₃ | CH₂ | 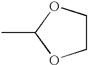 | | |
| 377 | CH₃ | (CH₂)₂ | 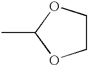 | | |
| 378 | H | CH₂ | 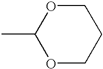 | | |
| 379 | H | (CH₂)₂ | 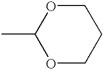 | | |
| 380 | CH₃ | CH₂ | 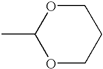 | | |
| 381 | CH₃ | (CH₂)₂ | 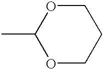 | | |
| 382 | H | CH₂ | 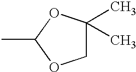 | | |
| 383 | CH₃ | CH₂ | 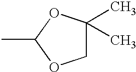 | | |
| 384 | H | CH₂ | 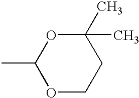 | | |
| 385 | CH₃ | CH₂ | 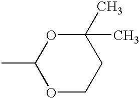 | | |
| 386 | H | CH₂ | 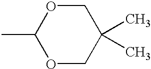 | | |
| 387 | CH₃ | CH₂ | 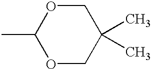 | | |

TABLE 3

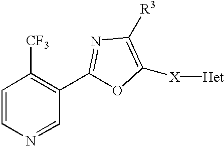

| Ex. No. | R³ | X | Het | m.p. [° C.] | Note |
|---|---|---|---|---|---|
| 388 | H | CH₂ | 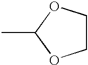 | | |
| 389 | H | (CH₂)₂ | 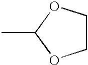 | | |
| 390 | CH₃ | CH₂ | 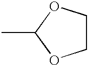 | | |
| 391 | CH₃ | (CH₂)₂ | 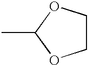 | | |
| 392 | H | CH₂ | 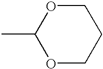 | | |
| 393 | CH₃ | (CH₂)₂ | 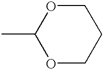 | | |
| 394 | CH₃ | CH₂ | 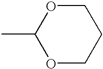 | | |
| 395 | CH₃ | (CH₂)₂ | 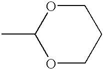 | | |
| 396 | H | CH₂ | 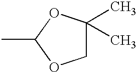 | | |
| 397 | CH₃ | CH₂ | 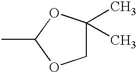 | | |
| 398 | H | CH₂ | 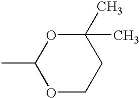 | | |
| 399 | CH₃ | CH₂ | 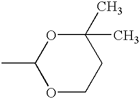 | | |
| 400 | H | CH₂ | 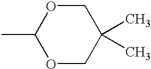 | | |
| 401 | CH₃ | CH₂ | 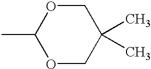 | | |

### B. Formulation Examples

a) A dust is obtained by mixing 10 parts by weight of active substance and 90 parts by weight of talc as inert substance and comminuting the mixture in a hammermill.

b) A wettable powder which is readily dispersible in water is obtained by mixing 25 parts by weight of active substance, 65 parts by weight of kaolin-containg quartz as inert substance, 10 parts by weight of potassium lignosulfonate and 1 part by weight of sodium oleoylmethyltaurinate as wetter and dispersant and grinding the mixture in a pinned-disk mill.

c) A dispersion concentrate which is readily dispersible in water is prepared by mixing 40 parts by weight of active substance with 7 parts by weight of a sulfosuccinic aminoester, 2 parts by weight of a sodium lignosulfonate and 51 parts by weight of water and grinding the mixture in a ball mill to a fineness of below 5 microns.

d) An emulsifiable concentrate can be prepared from 15 parts by weight of active substance, 75 parts by weight of cyclohexane as solvent and 10 parts by weight of oxethylated nonylphenol (10 EO) as emulsifier.

e) Granules can be prepared from 2 to 15 parts by weight of active substance and an inert granule carrier material such as attapulgite, pumice granules and/or quartz sand. It is expedient to use a suspension of the wettable powder of Example b) with a solids content of 30% and spray this onto the surface of attapulgite granules, dry the material and mix it intimately. The wettable powder amounts to approximately 5% by weight and the inert carrier material to approximately 95% by weight of the finished granules.

### BIOLOGICAL EXAMPLES

### Example 1

Pregerminated field bean seeds (Vicia faba) with seminal roots were transferred into amber glass bottles filled with tap water and then populated with approximately 100 black bean aphids (Aphis fabae). The plants and aphids were then immersed for 5 seconds in an aqueous solution of the formulated preparation to be tested. After the solution had been allowed to run off, the plants and animals were stored in a controlled-environment cabinet (16 hours light/day, 25° C., 40-60% atmospheric humidity). After storage for 3 and 6 days, the effect of the preparation on the aphids was determined. At a concentration of 300 ppm (based on the active substance content), the preparations of Examples No. 1, 8, 20, 28, 30, 34, 36, 40, 52, 54, 56, 58, 118, 138, 146, 148, 154, 158, 176,184,369 resulted in 90-100% mortality of the aphids.

### Example 2

Pregerminated field bean seeds (Vicia faba) with seminal roots were transferred into amber glass bottles filled with tap water. Four milliliters of an aqueous solution of the formulated preparation to be tested were pipetted into the amber glass bottle. Then, the field bean was infested with approx. 100 black bean aphids (Aphis fabae). The plant and animals were then stored in a controlled-environment cabinet (16 hours light/day, 25° C., 40-60% atmospheric humidity). After storage for 3 and 6 days, the root-systemic action of the preparation on the aphids was determined. At a concentration of 300 ppm (based on active substance content), the preparations of Examples No. 1, 8, 20, 28, 30, 34, 36, 40, 52, 54, 56, 58, 118, 138, 146, 148, 154, 158, 176, 184, 368, 369, 370 caused 90-100% mortality of the aphids owing to the root-systemic activity.

We claim:

1. An heterocyclylalkylazole of the formula (I), where appropriate also as salt, 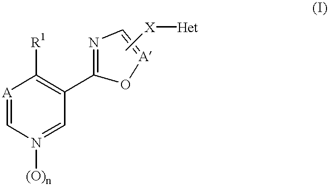
where the symbols and indices are defined as follows:
R¹ is (C₁-C₄)haloalkyl;
A, A′ are identical or different and are CH or N; where, in the event that A′=CH, the oxazole ring optionally has attached to it a further substituent;
n is 0 or 1;
X is a direct bond or an unbranched or branched (C₁-C₈)alkanediyl group in which a group 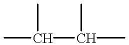
is optionally replaced by 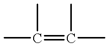
and/or a group —CH₂—CH₂— is optionally replaced by —C≡C— and in which a CH₂ group is optionally replaced by a carbonyl group or a heteroatom unit;
R^{x} is H, (C₁-C₄)alkyl, (C₂-C₄)alkenyl, (C₂-C₄)alkynyl, (Ci-C₄)alkoxycarbonyl, (C₁-C₄)alkanoyl, (C₁-C₄)alkylsulfonyl, (C₁-C₄)alkoxy-(C₁-C₄)alkyl or (C₁-C₄)alkoxy;
Het is a group of the general formula (II) 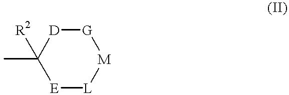
which is optionally mono- or polysubstituted and in which the symbols and indices have the following meanings:
D and E are identical or different and are in each case oxygen, S(O)ₚ where p=0, 1 or 2 or NR^{y},
R^{y} is R^{x}, aryl, aryl-(C₁-C₄)alkyl;
G and L are identical or different and are in each case CH₂ or a carbonyl group;
M is a direct bond, (C₁-C₃)alkanediyl, —CH═CH—, 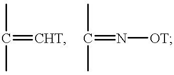
T is hydrogen, (C₁-C₈)alkyl, (C₁-C₈)haloalkyl, (C₂-C₈)alkenyl, (C₂-C₈)alkynyl, (C₃-C₈)cycloalkyl, aryl, aryl-(C₁-C₄)alkyl;
it being possible for the aromatic ring systems mentioned for R^{y} and T to be unsubstituted or to be provided by up to three, in the case of fluorine also up to the maximum number of, identical or different substituents.

2. A compound as claimed in claim 1, wherein
R¹ fluoroalkyl,
n is 0 and
A is CH.

3. A compound as claimed in claim 2, wherein
R¹ is trifluoromethyl.

4. A compound as claimed in claim 1, wherein
A′ is nitrogen.

5. A compound as claimed in claims 1, wherein
D and E are oxygen,
G and L are CH₂ and
M is a direct bond or CH₂.

6. An insecticidally, acaricidally and nematicidally active composition comprising at least one compound of the formula (I) as claimed in claim 1.

7. An insecticidally, acaricidally and nematicidally active composition as claimed in claim 6 in a mixture with carriers and/or surfactants.

8. A composition as claimed in claim 6 which comprises an additional active substance from the group of the acaricides, fungicides, herbicides, insecticides, nematicides or growth regulators.

9. An animal health medicine comprising a composition as claimed in claim 6 for the preparation of an animal medicine.

10. A method of controlling harmful insects, Acarina and nematodes, which comprises applying an effective amount of a compound as claimed in claim 1 to the site where the action is desired.

11. A method of protecting useful plants against the undesired effects of harmful insects, Acarina and nematodes, which comprises applying at least one of the compounds as claimed in claim 1 for treating the seed of the useful plants.
